SEQUENCE LISTING (23 sequences)

SEQ ID NO: 1 - protein, 552 aa, Artificial Sequence
  Kuma062-M
SDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARRRFRMQRRAEGGFEARSQAAAPTAYTPLDVAQAYQFPEGLDGQGQCIAIIELGGGYDEASLAQYFASLGVPAPQVVSVSVDGASNQPTGDPEGPDGEVTLDIEVAGALAPGAKFAVYFAPDTTAGFLDAITTAIHDPTLKPSVVSISWSMPEDSWTSAAIAAMNRAFLDAAALGVTVLAAAGDQGSTSGEQDGLYHVHFPAASPYVLACGGTRLVASGGRIAQETVWNQGPDGGATGGGVSRIFPLPAWQEHANVPPSANPGASSGRGVPDLAGNADPQTGYEVVIDGEATVTGGTSAVAPLFAALVARINQKLGKAVGYLNPTLYQLPADVFHDITEGNNDIANRAQIYQAGPGWDPCTGLGSPIGVRLLQALLPSASQPQP

SEQ ID NO: 2 - protein, 189 aa, Artificial Sequence
  Kuma011 Pre-Protein Domain
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARPHFRMQRRAEGGFEARSQA

SEQ ID NO: 3 - protein, 364 aa, Artificial Sequence
  Kuma011 Mature Peptide
AAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQP

SEQ ID NO: 4 - protein, 573 aa, Artificial Sequence
  Kuma010 (Full Length)
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 5 - protein, 384 aa, Artificial Sequence
  Kuma010 Mature Peptide
AAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQPGSTENLYFQSGALEHHHHHH

SEQ ID NO: 6 - protein, 553 aa, Artificial Sequence
  Kuma011 (Full Length)
MSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARPHFRMQRRAEGGFEARSQAAAPTAYTPLDVAQAYQFPEGLDGQGQCIAIIELGGGYDEASLAQYFASLGVPAPQVVSVSVDGASNQPTGDPKGPDGEVELDIEVAGALAPGAKFAVYFAPDTTAGFLDAITTAIHDPTLKPSVVSISWSGPEDSWTSAAIAAMNRAFLDAAALGVTVLAAAGDSGSTGGEQDGLYHVHFPAASPYVLACGGTRLVASGGRIAQETVWNDGPDGGATGGGVSRIFPLPAWQEHANVPPSANPGASSGRGVPDLAGNADPATGYEVVIDGEATVIGGTSAVAPLFAALVARINQKLGKAVGYLNPTLYQLPADVFHDITEGNNDIANRAQIYQAGPGWDPCTGLGSPIGVRLLQALLPSASQPQP

SEQ ID NO: 7 - protein, 188 aa, Artificial Sequence
  Kuma062-M Pre-Protein Domain
SDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARRRFRMQRRAEGGFEARSQA

SEQ ID NO: 8 - protein, 363 aa, Artificial Sequence
  Kuma062-M Mature Peptide
AAPTAYTPLDVAQAYQFPEGLDGQGQCIAIIELGGGYDEASLAQYFASLGVPAPQVVSVSVDGASNQPTGDPEGPDGEVTLDIEVAGALAPGAKFAVYFAPDTTAGFLDAITTAIHDPTLKPSVVSISWSMPEDSWTSAAIAAMNRAFLDAAALGVTVLAAAGDQGSTSGEQDGLYHVHFPAASPYVLACGGTRLVASGGRIAQETVWNQGPDGGATGGGVSRIFPLPAWQEHANVPPSANPGASSGRGVPDLAGNADPQTGYEVVIDGEATVTGGTSAVAPLFAALVARINQKLGKAVGYLNPTLYQLPADVFHDITEGNNDIANRAQIYQAGPGWDPCTGLGSPIGVRLLQALLPSASQPQ

SEQ ID NO: 9 - protein, 4 aa, Artificial Sequence
  gliadin
PQLP

SEQ ID NO: 10 - protein, 4 aa, Artificial Sequence
  gliadin
PQQP

SEQ ID NO: 11 - protein, 9 aa, Artificial Sequence
  gliadin
PFPQPQLPY

SEQ ID NO: 12 - protein, 9 aa, Artificial Sequence
  gliadin
PFPQPQQPF

SEQ ID NO: 13 - protein, 33 aa, Artificial Sequence
  Polypeptide
LQLQPFPQPQLPYPQPQLPYPQPQLPYPQPQPF

SEQ ID NO: 14 - protein, 26 aa, Artificial Sequence
  Polypeptide
FLQPQQPFPQQPQQPYPQQPQQPFPQ

SEQ ID NO: 15 - protein, 60 aa, Artificial Sequence
  cleavable histidine tag
  misc_feature (1)..(25): Xaa is any amino acid
  misc_feature (28)..(28): Xaa is  Lys or Gln
  misc_feature (30)..(55): Xaa is any amino acid
XXXXXXXXXXXXXXXXXXXXXXXXXPQXPXXXXXXXXXXXXXXXXXXXXXXXXXHHHHHH

SEQ ID NO: 16 - protein, 28 aa, Artificial Sequence
  cleavable histidine tag
GSSGSSGSQPQLPYGSSGSSGSHHHHHH

SEQ ID NO: 17 - protein, 20 aa, Artificial Sequence
  histidine tag
GSTENLYFQSGALEHHHHHH

SEQ ID NO: 18 - protein, 7 aa, Artificial Sequence
  TEV Protease Cut site
ENLYFQS

SEQ ID NO: 19 - protein, 5 aa, Artificial Sequence
  R5
QQPFP

SEQ ID NO: 20 - protein, 6 aa, Artificial Sequence
  G12
QPQLPY

SEQ ID NO: 21 - protein, 4 aa, Artificial Sequence
  gliadinase
SDME

SEQ ID NO: 22 - DNA, 1659 bp, Artificial Sequence
  A nucleic acid that encodes the Kuma062-M Protein
agtgatatggaaaaaccgtggaaagaaggtgaagaagcccgcgcagtgctgcaaggtcatgctcgtgcgcaggcaccgcaagcagtcgataaaggcccggtggcaggtgacgaacgcatggctgttaccgtggttctgcgtcgccagcgtgcaggtgaactggcggcccacgtggaacgtcaagcagctattgctccgcatgcgcgcgaacacctgaaacgtgaagcgtttgcggccagtcatggtgcgtccctggatgactttgccgaactgcgtcgcttcgcagatgctcacggcctggcgctggaccgtgcaaacgttgcagctggcaccgccgttctgtctggtccggacgatgcaatcaatcgcgcttttggtgtggaactgcgtcatttcgatcacccggacggctcatatcgttcgtacctgggtgaagtcaccgtgccggccagtattgcaccgatgatcgaagcggttctgggcctggatacgcgtccggtcgcccgccgtcgttttcgtatgcagcgtcgcgcagaaggcggtttcgaagctcgttcccaagcggcggcaccgaccgcatatacgccgctggatgttgcgcaggcctaccaatttccggaaggtctggacggccagggtcaatgcattgccattatcgaactgggcggtggctatgatgaagcttcactggcgcagtacttcgcgtcgctgggcgtgccggcaccgcaagtcgtgagtgtttccgtcgatggtgcgagcaaccagccgaccggtgatccggaaggtccggacggtgaagtgaccctggatatcgaagttgcaggcgctctggcgccgggtgccaaatttgcagtgtatttcgcgccggataccactgccggttttctggacgcgattaccacggccatccacgatccgacgctgaaaccgagcgttgtctcaatttcgtggagcatgccggaagacagctggacctctgctgcgatcgccgcaatgaaccgtgcgtttctggatgctgcggccctgggtgtgaccgttctggcagctgcgggcgaccagggttctacgagcggcgaacaggacggtctgtatcatgtgcatttcccggccgcatcaccgtacgttctggcgtgcggtggcacgcgcctggtcgcatcgggtggccgtattgcgcaggaaaccgtctggaaccagggtccggacggtggtgcaacgggtggcggtgtgagccgcatcttcccgctgccggcatggcaggaacacgctaacgttccgccgtctgcaaatccgggcgcgagcagcggccgtggtgtcccggatctggctggtaatgcggacccgcagaccggttatgaagtggttattgatggcgaagcaaccgtcaccggcggtacgagcgccgtggcaccgctgtttgctgcgctggttgcgcgtattaaccagaaactgggcaaagcagttggttatctgaatccgaccctgtaccaactgccggcagatgttttccatgacatcacggagggtaacaatgatattgcaaaccgtgcgcagatttatcaagcaggtccgggctgggacccgtgtaccggtctgggttcaccgattggtgtgcgtctgctgcaagcactgttgccgagtgcctcccagccgcaaccgtga

SEQ ID NO: 23 - protein, 553 aa, Artificial Sequence
  Polypeptide
  misc_feature (1)..(1): Wherein Xaa is not Met.
XSDMEKPWKEGEEARAVLQGHARAQAPQAVDKGPVAGDERMAVTVVLRRQRAGELAAHVERQAAIAPHAREHLKREAFAASHGASLDDFAELRRFADAHGLALDRANVAAGTAVLSGPDDAINRAFGVELRHFDHPDGSYRSYLGEVTVPASIAPMIEAVLGLDTRPVARRRFRMQRRAEGGFEARSQAAAPTAYTPLDVAQAYQFPEGLDGQGQCIAIIELGGGYDEASLAQYFASLGVPAPQVVSVSVDGASNQPTGDPEGPDGEVTLDIEVAGALAPGAKFAVYFAPDTTAGFLDAITTAIHDPTLKPSVVSISWSMPEDSWTSAAIAAMNRAFLDAAALGVTVLAAAGDQGSTSGEQDGLYHVHFPAASPYVLACGGTRLVASGGRIAQETVWNQGPDGGATGGGVSRIFPLPAWQEHANVPPSANPGASSGRGVPDLAGNADPQTGYEVVIDGEATVTGGTSAVAPLFAALVARINQKLGKAVGYLNPTLYQLPADVFHDITEGNNDIANRAQIYQAGPGWDPCTGLGSPIGVRLLQALLPSASQPQP

(19) United States
(12) Patent Application Publication
(10) Pub. No.: US 2025/0064903 A1
(43) Pub. Date: Feb. 27, 2025

(54) COMPOSITIONS AND METHODS FOR TREATING CELIAC SPRUE DISEASE

(71) Applicants: University of Washington, Seattle, WA (US); THE REGENTS OF THE UNIVERSITY OF CALIFORNIA, Oakland, CA (US)

(72) Inventors: Ingrid Swanson PULTZ, Seattle, WA (US); Clancey WOLF, Seattle, WA (US); Justin Bloomfield SIEGEL, Seattle, WA (US); Christine Elaine TINBERG, Seattle, WA (US); Lance STEWART, Seattle, WA (US); David BAKER, Seattle, WA (US)

(21) Appl. No.: 18/034,622

(22) PCT Filed: Oct. 29, 2021
(86) PCT No.: PCT/US2021/057197

§ 371 Date: Apr. 28, 2023

Related U.S. Application Data
(60) Provisional application No. 63/108,163, filed on Oct. 30, 2020.

(51) Int. Cl. A61K 38/48; C12N 9/52

(52) U.S. Cl. CPC A61K 38/488; C12N 9/52

(57) ABSTRACT

The present disclosure is directed to polypeptides capable of cleaving gluten proteins, e.g., gliadins, nucleic acid molecules encoding the same, pharmaceutical compositions comprising the same, and methods of use thereof for treating celiac sprue disease and/or non-celiac gluten sensitivity (NCGS).

### RELATED APPLICATIONS

This application is a U.S. national phase of International Application No. PCT/US2021/057197, filed on Oct. 29, 2021, which claims priority to U.S. Provisional Application Ser. No. 63/108,163, filed Oct. 30, 2020, both of which are incorporated by reference herein in their entirety.

### REFERENCE TO THE SEQUENCE LISTING

The instant application contains a Sequence Listing which has been submitted electronically in ASCII format and is hereby incorporated by reference in its entirety. Said ASCII copy, created on Jul. 17, 2024, is named 20-1718-WO-US_ST25.txt and is 38,925 bytes in size.

### FIELD OF DISCLOSURE

The present disclosure relates to compositions capable of cleaving gluten peptides, e.g., gliadins, and the use thereof in the treatment of gluten sensitivity, including celiac sprue disease.

### BACKGROUND

Celiac sprue is a highly prevalent disease in which dietary proteins found in wheat, barley, and rye products known as “glutens” evoke an immune response in the small intestine of genetically predisposed individuals. The resulting inflammation can lead to the degradation of the villi of the small intestine, impeding the absorption of nutrients. Symptoms can appear in early childhood or later in life, and range widely in severity, from diarrhea, fatigue and weight loss to abdominal distension, anemia, and neurological symptoms. There are currently no effective therapies for this lifelong disease except the total elimination of glutens from the diet. Although celiac sprue remains largely underdiagnosed, its prevalence in the US and Europe is estimated at 0.5-1.0% of the population. In addition to celiac sprue, a significant fraction of the population is thought to suffer from the condition of non-celiac gluten sensitivity (NCGS), which is caused by the ingestion of gluten but is mechanistically distinct from celiac disease, though the symptoms are frequently indistinguishable from those of celiac sprue. The identification of suitable naturally-occurring enzymes as oral therapeutics for celiac disease and NCGS is difficult due to the stringent physical and chemical requirements to specifically and efficiently degrade gluten-derived peptides in the harsh and highly acidic environment of the human digestive tract. Since gluten peptides initiate the immune response immediately upon entering the intestines, it is imperative that any oral enzyme therapeutic for celiac disease break down these immunogenic gluten regions in the gastric compartment, thereby preventing these gluten peptides from causing intestinal damage due to inflammation.

### SUMMARY OF THE DISCLOSURE

Certain aspects of the present disclosure are directed to a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1. In some aspects, the polypeptide comprises an amino acid sequence having at least 85% sequence identity to the amino acid sequence set forth in SEQ ID NO: 1. In some aspects, the polypeptide comprises an amino acid sequence having at least 90% sequence identity to the amino acid sequence set forth in SEQ ID NO: 1. In some aspects, the polypeptide comprises an amino acid sequence having at least 95% sequence identity to the amino acid sequence set forth in SEQ ID NO: 1. In some aspects, the polypeptide comprises an amino acid sequence having at least 99% sequence identity to the amino acid sequence set forth in SEQ ID NO: 1. In some aspects, the polypeptide comprises the amino acid sequence set forth in SEQ ID NO: 1.

In some aspects, the amino acid residue corresponding to amino acid 467 of SEQ ID NO: 6 is a Ser. In some aspects, the amino acid residue corresponding to amino acid 267 of SEQ ID NO: 6 is a Glu. In some aspects, the amino acid residue corresponding to amino acid 271 of SEQ ID NO: 6 is an Asp.

In some aspects, the polypeptide is capable of cleaving gliadin.

Certain aspects of the present disclosure are directed to a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 8. In some aspects, the polypeptide comprises an amino acid sequence having at least 85% sequence identity to the amino acid sequence set forth in SEQ ID NO: 8.

In some aspects, the polypeptide comprises an amino acid sequence having at least 90% sequence identity to the amino acid sequence set forth in SEQ ID NO: 8. In some aspects, the polypeptide comprises an amino acid sequence having at least 95% sequence identity to the amino acid sequence set forth in SEQ ID NO: 8. In some aspects, the polypeptide comprises an amino acid sequence having at least 99% sequence identity to the amino acid sequence set forth in SEQ ID NO: 8. In some aspects, the polypeptide comprises the amino acid sequence set forth in SEQ ID NO: 8.

In some aspects, the amino acid residue corresponding to amino acid 278 of SEQ ID NO: 3 is a Ser. In some aspects, the amino acid residue corresponding to amino acid 78 of SEQ ID NO: 3 is a Glu. In some aspects, the amino acid residue corresponding to amino acid 82 of SEQ ID NO: 3 is an Asp.

In some aspects, the polypeptide is capable of cleaving gliadin.

Certain aspects of the present disclosure are directed to a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1; wherein the polypeptide comprises the amino acid sequence set forth in SEQ ID NO: 8. In some aspects, the polypeptide comprises an amino acid sequence having at least 85% sequence identity to the amino acid sequence set forth in SEQ ID NO: 1. In some aspects, the polypeptide comprises an amino acid sequence having at least 90% sequence identity to the amino acid sequence set forth in SEQ ID NO: 1. In some aspects, the polypeptide comprises an amino acid sequence having at least 95% sequence identity to the amino acid sequence set forth in SEQ ID NO: 1. In some aspects, the polypeptide comprises an amino acid sequence having at least 99% sequence identity to the amino acid sequence set forth in SEQ ID NO: 1. In some aspects, the polypeptide comprises the amino acid sequence set forth in SEQ ID NO: 1.

In some aspects, the amino acid residue corresponding to amino acid 467 of SEQ ID NO: 6 is a Ser. In some aspects, the amino acid residue corresponding to amino acid 267 of SEQ ID NO: 6 is a Glu. In some aspects, the amino acid residue corresponding to amino acid 271 of SEQ ID NO: 6 is an Asp.

In some aspects, the polypeptide is capable of cleaving gliadin.

In some aspects, the polypeptide comprises a histidine tag, wherein the histidine tag is fused at the C-terminus of the polypeptide. In some aspects, the histidine tag comprises the amino acid sequence set forth in SEQ ID NO: 17 (GSTENLYFQSGALEHHHHHH). In some aspects, the histidine tag comprises a cleavable histidine tag, including but not limited to a cleavable histidine tag comprising the amino acid sequence set forth in SEQ ID NO: 15 (X_{N}PQ(L/Q)PX_{N}HHHHHH), wherein X_{N} is an linker of between 1-25 amino acid residues. In some aspects, the cleavable histidine tag comprises the amino acid sequence set forth in SEQ ID NO: 16 (GSSGSSGSQPQLPYGSSGSSGSHHHHHH).

Certain aspects of the present disclosure are directed to a nucleic acid molecule encoding a polypeptide disclosed herein.

Certain aspects of the present disclosure are directed to a nucleic acid expression vector comprising a nucleic acid molecule disclosed herein.

Certain aspects of the present disclosure are directed to a recombinant host cell comprising a nucleic acid molecule or a nucleic acid expression vector disclosed herein.

Certain aspects of the present disclosure are directed to a pharmaceutical composition, comprising a polypeptide disclosed herein, a nucleic acid molecule disclosed herein, a nucleic acid expression vector disclosed herein, a recombinant host cell disclosed herein, or any combination thereof and a pharmaceutically acceptable carrier.

Certain aspects of the present disclosure are directed to a method for treating celiac sprue or non-celiac gluten sensitivity (NCGS), comprising administering to an individual with celiac sprue or NCGS an amount effective to treat the celiac sprue or NCGS of a polypeptide disclosed herein, a nucleic acid molecule disclosed herein, a nucleic acid expression vector disclosed herein, a recombinant host cell disclosed herein, or a pharmaceutical composition disclosed herein. In some aspects, the polypeptide, the nucleic acid molecule, the nucleic acid expression vector, the recombinant host cell, or the pharmaceutical composition is administered orally.

In some aspects, the present disclosure is directed to a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1, wherein the first amino acid at the N-terminus of the polypeptide is a Ser (S). In some aspects, the polypeptide has gliadinase activity.

In some aspects, the present disclosure is directed to a polypeptide comprising an amino acid sequence having at least about 75%, at least about 80%, at least about 85%, at least about 90%, at least about 95%, at least about 96%, at least about 98%, at least about 99%, or about 100% sequence identity to the amino acid sequence set forth in SEQ ID NO: 1, wherein the polypeptide does not comprise a Met (M) at the N-terminus of the polypeptide.

In some aspects, the present disclosure is directed to a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 23 wherein the Xaa in SEQ ID NO: 23 is not a Met (M).

In some aspects, the present disclosure is directed to a polypeptide comprising an amino acid sequence an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1, wherein the first amino acid at the N-terminus of the polypeptide is a Ser (S): wherein the polypeptide comprises the amino acid sequence set forth in SEQ ID NO: 8.

In some aspects, the first two N-terminal amino acids of the polypeptide, from N-terminus to C-terminus, are Ser-Asp (SD). In some aspects, the first three N-terminal amino acids of the polypeptide, from N-terminus to C-terminus, are Ser-Asp-Met (SDM). In some aspects, the first four N-terminal amino acids of the polypeptide, from N-terminus to C-terminus, are Ser-Asp-Met-Glu (SDME).

In some aspects, the polypeptide disclosed herein comprises an amino acid sequence having at least 85% sequence identity to the amino acid sequence set forth in SEQ ID NO: 1. In some aspects, the polypeptide disclosed herein comprises an amino acid sequence having at least 90% sequence identity to the amino acid sequence set forth in SEQ ID NO: 1. In some aspects, the polypeptide disclosed herein comprises an amino acid sequence having at least 95% sequence identity to the amino acid sequence set forth in SEQ ID NO: 1. In some aspects, the polypeptide disclosed herein comprises an amino acid sequence having at least 99% sequence identity to the amino acid sequence set forth in SEQ ID NO: 1. In some aspects, the polypeptide disclosed herein comprises the amino acid sequence set forth in SEQ ID NO: 1.

In some aspects of the polypeptide disclosed herein, the amino acid residue corresponding to amino acid 467 of SEQ ID NO: 1 is a Ser. In some aspects of the polypeptide disclosed herein, the amino acid residue corresponding to amino acid 267 of SEQ ID NO: 1 is a Glu. In some aspects of the polypeptide disclosed herein, the amino acid residue corresponding to amino acid 271 of SEQ ID NO: 1 is an Asp.

In some aspects of the present disclosure, the polypeptide is capable of cleaving gliadin. In some aspects, the polypeptide has improved enzymatic activity as compared to Kuma011.

In some aspects, the polypeptide disclosed herein further comprises a histidine tag, wherein the histidine tag is fused at the C-terminus of the polypeptide. In some aspects, the histidine tag comprises the amino acid sequence set forth in SEQ ID NO: 17 (GSTENLYFQSGALEHHHHHH). In some aspects, the histidine tag comprises a cleavable histidine tag, including but not limited to a cleavable histidine tag comprising the amino acid sequence set forth in SEQ ID NO: 15 (XNPQ(L/Q)PXNHHHHHH), wherein XN is an linker of between 1-25 amino acid residues. In some aspects, the cleavable histidine tag comprises the amino acid sequence set forth in SEQ ID NO: 16 (GSSGSSGSQPQLPYGSSGSSGSHHHHHH).

In some aspects, the present disclosure is directed to a nucleic acid molecule encoding the polypeptide described herein. In some aspects, the present disclosure is directed to a nucleic acid expression vector comprising the nucleic acid molecule described herein.

In some aspects, the present disclosure is directed to a recombinant host cell comprising the nucleic acid molecule or the nucleic acid expression vector described herein. In some aspects, the host cell is prokaryotic. In some aspects, the host cell is eukaryotic.

In some aspects, the present disclosure is directed to a pharmaceutical composition, comprising the polypeptide, the nucleic acid molecule the nucleic acid expression vector, or the recombinant host cell described herein, or any combination thereof and a pharmaceutically acceptable carrier.

In some aspects, the present disclosure is directed to a method for treating celiac sprue or non-celiac gluten sensitivity (NCGS) in a subject, comprising administering to the subject with celiac sprue or NCGS an amount effective to treat the celiac sprue or NCGS of the polypeptide, the nucleic acid molecule, the nucleic acid expression vector, the recombinant host cell, or the pharmaceutical composition described herein, thereby treating the celiac sprue or NCGS.

In some aspects, the present disclosure is directed to a method for reducing celiac sprue or non-celiac gluten sensitivity (NCGS) related inflammation in a subject, comprising administering to the subject with celiac sprue or NCGS an amount effective to reduce the celiac sprue or NCGS related inflammation of the polypeptide, the nucleic acid molecule, the nucleic acid expression vector, the recombinant host cell, or the pharmaceutical composition described herein, thereby reducing the inflammation. In some aspects, the polypeptide, the nucleic acid molecule, the nucleic acid expression vector, the recombinant host cell, or the pharmaceutical composition is administered orally.

In some aspects, the present disclosure is directed to a method for degrading gluten in a food item, comprising contacting the food item with an amount effective to degrade the gluten with the polypeptide or the pharmaceutical composition described herein, thereby degrading the gluten in the food item.

In some aspects, the present disclosure is directed to a method for degrading gliadin in a food item, comprising contacting the food item with an amount effective to degrade the gliadin with the polypeptide, or the pharmaceutical composition of described herein, thereby degrading the gliadin in the food item.

In some aspects, the method degrades at least about 75%, at least about 80%, at least about 85%, at least about 90%, at least about 95%, at least about 96%, at least about 98%, at least about 99%, or about 100% of the gluten or gliadin in the food item. In some aspects, the method degrades the gluten or gliadin in the food item in less than about 1.5 hour, less than about 1 hour, less than about 45 minutes, less than about 40 minutes, less than about 30 minutes, less than about 25 minutes, less than about 20 minutes, less than about 15 minutes, less than about 10 minutes, or less than about 5 minutes. In some aspects, the method degrades the gluten or gliadin in the food item under a pH value less than about 6.5, less than about 6.0, less than about 5.5, less than about 5.0, less than about 4.5, less than about 4.0, less than about 3.5, less than about 3.0, less than about 2.5, less than about 2.0, or less than about 1.5.

### DETAILED DESCRIPTION

The present disclosure provides gliadinases that are capable of degrading gliadin peptides. Some aspects of the present disclosure are directed to a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1, wherein the first amino acid at the N-terminus of the polypeptide is a Ser (S). In some aspects, the polypeptide does not comprise a Met (M) at the N-terminus of the polypeptide. In some aspects, the polypeptide comprises the amino acid sequence set forth in SEQ ID NO: 8.

### 1. Definitions

In order that the present disclosure may be more readily understood, certain terms are first defined. Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition.

In addition, it should be noted that whenever a value or range of values of a parameter are recited, it is intended that values and ranges intermediate to the recited values are also part of this disclosure.

As used herein, the singular forms “a”, “an” and “the” include plural referents unless the context clearly dictates otherwise. “And” as used herein is interchangeably used with “or” unless expressly stated otherwise. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” usually means within 10%, within 5%, or more preferably within 1%, of a given value or range.

The term “amino acid” refers to the twenty common naturally occurring amino acids. Naturally occurring amino acids include: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The terms “Celiac disease” and “celiac sprue disease” are used interchangeably and refer to a condition characterized by an inflammatory reaction to immunogenic peptides in gluten, the major protein in wheat flour, and to related proteins. Upon ingestion, α-gliadin is partially degraded by gastric and intestinal proteases to oligopeptides, referred to herein as “gliadins.” Gliadins are resistant to further proteolysis in gastric conditions due to their unusually high proline and glutamine content.

As used herein, a “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

As used herein, the terms “degrade” and “degradation” means to break down or decompose a target, e.g., a polypeptide, e.g., gluten, gliadins, and related proteins, into smaller oligopeptides. In certain embodiments, the degradation of a gliadin leads to the reduction and/or removal of the immunogenic peptides that are associated with celiac disease.

The term “gliadinase.” as used herein, refers to a polypeptide (enzyme) that can degrade one or more gliadins effectively. The term “gliadin,” as used herein, refers to proline (P)- and glutamine (Q)-rich peptide components of gluten. Exemplary gliadins comprises a PQLP (SEQ ID NO: 9) or PQQP (SEQ ID NO: 10) motif (such as PFPQPQLPY (SEQ ID NO: 11) and/or PFPQPQQPF (SEQ ID NO: 12)). In certain aspects, a gliadinase degrades one or more gliadins under acidic conditions, e.g., at pH 4 or lower.

The term “mutation,” as used herein, refers to insertion, deletion, or substitution of one or more amino acids in a polypeptide or of one or more nucleotides in a polynucleotide.

The term “variant,” as used herein, refers to a polypeptide or a polynucleotide that comprises one or more amino acid or nucleotide insertions, substitutions, or deletions relative to a reference polypeptide or a polynucleotide. In certain aspects, a variant polypeptide or polynucleotide has at least about 75% amino acid or nucleotide sequence identity, e.g., at least about 80%, at least about 85%, at least about 90%, at least about 91%, at least about 92%, at least about 93%, at least about 94%, at least about 95%, at least about 96%, at least about 97%, at least about 98%, at least about 99%, or about 100% sequence identity, to a reference polypeptide or polynucleotide sequence. In some aspects, a variant of a reference polypeptide or polynucleotide maintains one or more functions, activities, and/or structures of the reference polypeptide or polynucleotide. For example, a variant of a gliadinase disclosed herein maintains the function to degrade gluten and/or gliadin effectively. In another example, a variant of a polynucleotide encoding a gliadinase encodes a functional gliadinase.

Sequence identity is typically measured using sequence analysis software. Protein analysis software matches similar sequences using measures of similarity assigned to various substitutions, deletions, and other modifications, including conservative amino acid substitutions. For instance. GCG software contains programs such as Gap and Bestfit, which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another non-limiting example of algorithm that can be used to compare a sequence of the disclosure to a database containing a large number of sequences from different organisms is the computer program BLAST, e.g., BLASTP or TBLASTN, using default parameters. See, e.g., Altschul et al. (1990) J. Mol. Biol. 215:403-410) and Altschul et al. (1997) Nucleic Acids Res. 25:3389-402. each of which is incorporated by reference herein in its entirety.

As used herein, “treatment” or “treating” refers to an action that produces a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of damage, e.g., inflammation, that can lead to the degradation of the villi of the small intestine (including hyperplasia and villous atrophy), which characterizes celiac sprue or non-celiac gluten sensitivity (NCGS). Effective treatment may refer to alleviation or prevention of at least one symptom of celiac sprue or NCGS. Such effective treatment may reduce intraintestinal and/or extraintenstinal clinical manifestations of the celiac sprue or NCGS such as, e.g., diarrhea, abdominal pain, malnutrition, anemia, osteoporosis or any known symptom, inhibiting worsening of symptoms; limiting or preventing recurrence of celiac sprue in patients that have previously had the disorder; limiting or preventing recurrence of symptoms in patients that were previously symptomatic for celiac sprue or NCGS: and/or limiting development of celiac sprue or NCGS in a subject at risk of developing celiac sprue or NCGS, or not yet showing the clinical effects of celiac sprue or NCGS.

In some aspects, the treatment reduces inflammation in the small intestine. Effective reduction of inflammation can comprise a reduction of inflammation by at least about 1%, at least about 5%, at least about 10%, at least about 20%, at least about 30%, at least about 40%, at least about 50%, at least about 60%, at least about 70%, at least about 80%, at least about 90%, at least about 95%, at least about 99%, or about 100%, as compared to inflammation prior to treatment. Reduction of inflammation can be measured by any means.

Any individual experiencing a sensitivity to gluten can be treated according to the methods of the disclosure. In some aspects, the individual is suffering from celiac sprue. In some aspects, the individual is suffering from NCGS, In some aspects, the individual is a human subject. In some aspects, the individual is experiencing one or more symptoms related to gluten sensitivity. In some aspects, the individual is asymptomatic.

As used herein, an “amount effective” refers to an amount of the polypeptide that is sufficient to elicit a decrease in the severity or frequency of one or more symptoms of gluten sensitivity, e.g., celiac sprue or NCGS.

Polypeptides disclosed herein can be formulated as a pharmaceutical composition, such as those disclosed above, and can be administered via any suitable route, including orally, parentally, by inhalation spray, or topically in dosage unit formulations containing conventional pharmaceutically acceptable carriers, adjuvants, and vehicles.

All aspects of the disclosure can be used in combination, unless the context clearly dictates otherwise. All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique. 2^{nd} Ed. (R.I. Freshney. 1987. Liss, Inc. New York, NY), Gene Transfer and Expression Protocols, pp. 109-128, ed. E.J. Murray, The Humana Press Inc., Clifton, N.J.), and the Ambion 1998 Catalog (Ambion, Austin, TX).

### 2. Compositions of the Disclosure

The present disclosure provides gliadinases that effectively degrade gliadin. The present disclosure is based upon, at least partially, the discovery that various polypeptides containing one or more mutations relative to Kuma011, as described herein, have improved properties relative to Kuma011 and other known gliadinases such as SC-PEP (Sphingomonas capsulate peptidase) and endoprotease EPB2, including increased gliadin degradation activity. In certain embodiments, various polypeptides describes herein have improved gliadinase activity over Kuma011 and other known gliadinases under acidic condition.

In some aspects, the present disclosure provides polypeptides comprising an amino acid sequence at least 75% identical to the amino acid sequence set forth in SEQ ID NO:6, wherein (a) residue 467 is Ser, residue 267 is Glu, and residue 271 is Asp: and (b) the polypeptide comprises an amino acid substitution relative to SEQ ID NO: 6 at one or more residues selected from the group consisting of 221, 262E, 268, 269, 270, 319A, 320, 354E/Q/R/Y, 358S/Q/T, 368F/Q, 399, 402, 406, 424, 449, 461, 463, 105, 171, 172, 173, 174, and 456. In some aspects, the polypeptide comprises an amino acid substitution relative to SEQ ID NO: 6 at one or more residues selected from the group consisting of 221, 262E, 268, 269, 270, 319A, 320, 354E/Q/R/Y, 358S/Q/T, 368F/Q, 399, 402, 406, 424, 449, 461, and 463.

TABLE 1
Kuma Sequences

| | |
|---|---|
| Kuma 011 | MSDMEKPWKEGEEARAVLQGHARAQAPQAVDKGPVAGDERMAVTVVLRRQRAGELAAHV |
| (Full Length) | ERQAAIAPHAREHLKREAFAASHGASLDDFAELRRFADAHGLALDRANVAAGTAVLSGP |
| SEQ ID NO: 6 | DDAINRAFGVELRHFDHPDGSYRSYLGEVTVPASIAPMIEAVLGLDTRPVARPHERMQR |
| (Bold = Pre- | RAEGGFEARSQAAAPTAYTPLDVAQAYQFPEGLDGQGQCIAIIELGGGYDEASLAQYFA |
| protein | SLGVPAPQVVSVSVDGASNQPTGDPKGPDGEVELDIEVAGALAPGAKFAVYFAPDTTAG |
| domain) | FLDAITTAIHDPTLKPSVVSISWSGPEDSWTSAAIAAMNRAFLDAAALGVTVLAAAGDS |
| | GSTGGEQDGLYHVHFPAASPYVLACGGTRLVASGGRIAQETVWNDGPDGGATGGGVSRI |
| | FPLPAWQEHANVPPSANPGASSGRGVPDLAGNADPATGYEVVIDGEATVIGGTSAVAPL |
| | FAALVARINQKLGKAVGYLNPTLYQLPADVFHDITEGNNDIANRAQIYQAGPGWDPCTG |
| | LGSPIGVRLLQALLPSASQPQP |
| Kuma011 Pre- | MSDMEKPWKEGEEARAVLQGHARAQAPQAVDKGPVAGDERMAVTVVLRRQRAGELAAHV |
| Protein Domain | ERQAAIAPHAREHLKREAFAASHGASLDDFAELRRFADAHGLALDRANVAAGTAVLSGP |
| SEQ ID NO: 2 | DDAINRAFGVELRHFDHPDGSYRSYLGEVTVPASIAPMIEAVLGLDTRPVARPHERMQR |
| | RAEGGFEARSQA |
| Kuma011 Mature | AAPTAYTPLDVAQAYQFPEGLDGQGQCIAIIELGGGYDEASLAQYFASLGVPAPQVVSV |
| Peptide | SVDGASNQPTGDPKGPDGEVELDIEVAGALAPGAKFAVYFAPDTTAGELDAITTAIHDP |
| SEQ ID NO: 3 | TLKPSVVSISWSGPEDSWTSAAIAAMNRAFLDAAALGVTVLAAAGDSGSTGGEQDGLYH |
| | VHFPAASPYVLACGGTRLVASGGRIAQETVWNDGPDGGATGGGVSRIFPLPAWQEHANV |
| | PPSANPGASSGRGVPDLAGNADPATGYEVVIDGEATVIGGTSAVAPLFAALVARINQKL |
| | GKAVGYLNPTLYQLPADVFHDITEGNNDIANRAQIYQAGPGWDPCTGLGSPIGVRLLQA |
| | LLPSASQPQP |
| Kuma 010 (Full | MSDMEKPWKEGEEARAVLQGHARAQAPQAVDKGPVAGDERMAVTVVLRRQRAGELAAHV |
| Length) | ERQAAIAPHAREHLKREAFAASHGASLDDFAELRRFADAHGLALDRANVAAGTAVLSGP |
| SEQ ID NO: 4 | DDAINRAFGVELRHFDHPDGSYRSYLGEVTVPASIAPMIEAVLGLDTRPVARPHERMQR |
| (Bold = Pre- | RAEGGFEARSQAAAPTAYTPLDVAQAYQFPEGLDGQGQCIAIIELGGGYDEASLAQYFA |
| protein | SLGVPAPQVVSVSVDGASNQPTGDPKGPDGEVELDIEVAGALAPGAKFAVYFAPDTTAG |
| domain) | FLDAITTAIHDPTLKPSVVSISWSGPEDSWTSAAIAAMNRAFLDAAALGVTVLAAAGDS |
| | GSTGGEQDGLYHVHFPAASPYVLACGGTRLVASGGRIAQETVWNDGPDGGATGGGVSRI |
| | FPLPAWQEHANVPPSANPGASSGRGVPDLAGNADPATGYEVVIDGEATVIGGTSAVAPL |
| | FAALVARINQKLGKAVGYLNPTLYQLPADVFHDITEGNNDIANRAQIYQAGPGWDPCTG |
| | LGSPIGVRLLQALLPSASQPQPGSTENLYFQSGALEHHHHHH |
| Kuma010 Mature | AAPTAYTPLDVAQAYQFPEGLDGQGQCIAIIELGGGYDEASLAQYFASLGVPAPQVVSV |
| Peptide | SVDGASNQPTGDPKGPDGEVELDIEVAGALAPGAKFAVYFAPDTTAGFLDAITTAIHDP |
| SEQ ID NO: 5 | TLKPSVVSISWSGPEDSWTSAAIAAMNRAFLDAAALGVTVLAAAGDSGSTGGEQDGLYH |
| | VHFPAASPYVLACGGTRLVASGGRIAQETVWNDGPDGGATGGGVSRIFPLPAWQEHANV |
| | PPSANPGASSGRGVPDLAGNADPATGYEVVIDGEATVIGGTSAVAPLFAALVARINQKL |
| | GKAVGYLNPTLYQLPADVFHDITEGNNDIANRAQIYQAGPGWDPCTGLGSPIGVRLLQA |
| | LLPSASQPQPGSTENLYFQSGALEHHHHHH |
| Kuma 062-M | SDMEKPWKEGEEARAVLQGHARAQAPQAVDKGPVAGDERMAVTVVLRRQRAGELAAHVE |
| (Full Length) | RQAAIAPHAREHLKREAFAASHGASLDDFAELRRFADAHGLALDRANVAAGTAVLSGPD |
| SEQ ID NO: 1 | DAINRAFGVELRHFDHPDGSYRSYLGEVTVPASIAPMIEAVLGLDTRPVARRRFRMQRR |
| (Bold = Pre- | AEGGFEARSQAAAPTAYTPLDVAQAYQFPEGLDGQGQCIAIIELGGGYDEASLAQYFAS |
| protein | LGVPAPQVVSVSVDGASNQPTGDPEGPDGEVTLDIEVAGALAPGAKFAVYFAPDTTAGE |
| domain) | LDAITTAIHDPTLKPSVVSISWSMPEDSWTSAAIAAMNRAFLDAAALGVTVLAAAGDQG |
| | STSGEQDGLYHVHFPAASPYVLACGGTRLVASGGRIAQETVWNQGPDGGATGGGVSRIF |
| | PLPAWQEHANVPPSANPGASSGRGVPDLAGNADPQTGYEVVIDGEATVTGGTSAVAPLE |
| | AALVARINQKLGKAVGYLNPTLYQLPADVFHDITEGNNDIANRAQIYQAGPGWDPCTGL |
| | GSPIGVRLLQALLPSASQPQP |
| Kuma062-M Pre- | SDMEKPWKEGEEARAVLQGHARAQAPQAVDKGPVAGDERMAVTVVLRRQRAGELAAHVE |
| Protein Domain | RQAAIAPHAREHLKREAFAASHGASLDDFAELRRFADAHGLALDRANVAAGTAVLSGPD |
| SEQ ID NO: 7 | DAINRAFGVELRHFDHPDGSYRSYLGEVTVPASIAPMIEAVLGLDTRPVARRRERMQRR |
| | AEGGFEARSQA |
| Kuma062-M | AAPTAYTPLDVAQAYQFPEGLDGQGQCIAIIELGGGYDEASLAQYFASLGVPAPQVVSV |
| Mature Peptide | SVDGASNQPTGDPEGPDGEVTLDIEVAGALAPGAKFAVYFAPDTTAGELDAITTAIHDP |
| SEQ ID NO: 8 | TLKPSVVSISWSMPEDSWTSAAIAAMNRAFLDAAALGVTVLAAAGDQGSTSGEQDGLYH |
| | VHFPAASPYVLACGGTRLVASGGRIAQETVWNQGPDGGATGGGVSRIFPLPAWQEHANV |
| | PPSANPGASSGRGVPDLAGNADPQTGYEVVIDGEATVTGGTSAVAPLFAALVARINQKL |
| | GKAVGYLNPTLYQLPADVFHDITEGNNDIANRAQIYQAGPGWDPCTGLGSPIGVRLLQA |
| | LLPSASQPQ |

Kuma010. as referenced herein, comprises Kuma011 linked by an amino bond to a histidine tag sequence GSTENLYFQSGALEHHHHHH (SEQ ID NO: 17) at the C-terminus of the Kuma010) sequence.

Bold-face residues in the sequences provided in Table I represent the N-terminal portion present in the unprocessed polypeptide (i.e., which is cleaved off during processing); and non-bold faced font represents residues present in the processed version of the polypeptide (i.e., the mature peptide sequence). The numbers in parentheses indicate residue number: and where there are two numbers separated by a “/”, the number on the left is the residue number in the unprocessed version, and the number on the right is the residue number in the processed version. SEQ ID NO: 6 is the unprocessed version of Kuma011; SEQ ID NO: 3 is the processed version of Kuma011. As such, a polypeptide comprising the amino acid sequence set forth in SEQ ID NO: 6 (the full-length Kuma011 polypeptide) also necessarily comprises the amino acid sequence set forth in SEQ ID NO: 3 (the mature Kuma011 polypeptide). SEQ ID NO: 1 is the unprocessed version of Kuma062-M; and SEQ ID NO: 8 is the processed version of Kuma062-M. As such a polypeptide comprising the amino acid sequence set forth in SEQ ID NO: 1 (the full-length Kuma062-M polypeptide) also necessarily comprises the amino acid sequence set forth in SEQ ID NO: 8 (the mature Kuma062-M polypeptide).

In some aspects, a gliadinase of the present disclosure has a serine (Ser or S) at its N-terminus. In some aspects, a gliadinase of the present disclosure has an SD motif at its N-terminus. In some aspects, a gliadinase of the present disclosure has an SDM motif at its N-terminus. In some aspects, a gliadinase of the present disclosure has an SDME (SEQ ID NO: 21) at its N-terminus. In such an aspect, the first amino acid (position 1 of the polypeptide from its N-terminus is S: the second amino acid (position 2 of the polypeptide from its N-terminus is D; the third amino acid (position 3 of the polypeptide from its N-terminus is M; and the fourth amino acid (position 4 of the polypeptide from its N-terminus is E. In some aspects, an oligopeptide is attached to the N-terminal S at its N-terminus, wherein the amino acid adjacent to S at its N-terminus is not a methionine (M).

In some aspects, the polypeptide (e.g., the gliadinase) comprises an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1. In some aspects, the polypeptide comprises the amino acid sequence set forth in SEQ ID NO: 1. In some aspects, the polypeptide comprises an amino acid sequence having at least about 75% sequence identity to the amino acid sequence set forth in SEQ ID NO: 1. In some aspects, the polypeptide comprises an amino acid sequence having at least about 80% sequence identity to the amino acid sequence set forth in SEQ ID NO: 1. In some aspects, the polypeptide comprises an amino acid sequence having at least about 85% sequence identity to the amino acid sequence set forth in SEQ ID NO: 1. In some aspects, the polypeptide comprises an amino acid sequence having at least about 90% sequence identity to the amino acid sequence set forth in SEQ ID NO: 1. In some aspects, the polypeptide comprises an amino acid sequence having at least about 95% sequence identity to the amino acid sequence set forth in SEQ ID NO: 1. In some aspects, the polypeptide comprises an amino acid sequence having at least about 96% sequence identity to the amino acid sequence set forth in SEQ ID NO: 1. In some aspects, the polypeptide comprises an amino acid sequence having at least about 97% sequence identity to the amino acid sequence set forth in SEQ ID NO: 1. In some aspects, the polypeptide comprises an amino acid sequence having at least about 98% sequence identity to the amino acid sequence set forth in SEQ ID NO: 1. In some aspects, the polypeptide comprises an amino acid sequence having at least about 99% sequence identity to the amino acid sequence set forth in SEQ ID NO: 1. In some aspects, the polypeptide comprises a Ser at the amino acid residue corresponding to amino acid 467 in SEQ ID NO: 1. In some aspects, the polypeptide comprises a Glu at the amino acid residue corresponding to amino acid 267 in SEQ ID NO: 1. In some aspects, the polypeptide comprises an Asp at the amino acid residue corresponding to amino acid 271 in SEQ ID NO: 1.

In some aspects, the polypeptide (e.g., gliadinase) comprises an amino acid sequence having at least about 75%, at least about 80%, at least about 85%, at least about 90%, at least about 95%, at least about 96%, at least about 97%, at least about 98%, at least about 99%. or about 100% sequence identity to the amino acid sequence set forth in SEQ ID NO: 8. In some aspects, the polypeptide comprises the amino acid sequence set forth in SEQ ID NO: 8. In some aspects, the polypeptide comprises an amino acid sequence having at least about 75% sequence identity to the amino acid sequence set forth in SEQ ID NO: 8. In some aspects, the polypeptide comprises an amino acid sequence having at least about 80% sequence identity to the amino acid sequence set forth in SEQ ID NO: 8. In some aspects, the polypeptide comprises an amino acid sequence having at least about 85% sequence identity to the amino acid sequence set forth in SEQ ID NO: 8. In some aspects, the polypeptide comprises an amino acid sequence having at least about 90% sequence identity to the amino acid sequence set forth in SEQ ID NO: 8. In some aspects, the polypeptide comprises an amino acid sequence having at least about 95% sequence identity to the amino acid sequence set forth in SEQ ID NO: 8. In some aspects, the polypeptide comprises an amino acid sequence having at least about 96% sequence identity to the amino acid sequence set forth in SEQ ID NO: 8. In some aspects, the polypeptide comprises an amino acid sequence having at least about 97% sequence identity to the amino acid sequence set forth in SEQ ID NO: 8. In some aspects, the polypeptide comprises an amino acid sequence having at least about 98% sequence identity to the amino acid sequence set forth in SEQ ID NO: 8. In some aspects, the polypeptide comprises an amino acid sequence having at least about 99% sequence identity to the amino acid sequence set forth in SEQ ID NO: 8. In some aspects, the polypeptide comprises a Ser at the amino acid residue corresponding to amino acid 278 in SEQ ID NO: 3. In some aspects. the polypeptide comprises a Glu at the amino acid residue corresponding to amino acid 78 in SEQ ID NO: 3. In some aspects, the polypeptide comprises an Asp at the amino acid residue corresponding to amino acid 82 in SEQ ID NO: 3.

In some aspects, the polypeptide (e.g., gliadinase) comprises an amino acid sequence having at least about 75%, at least about 80%, at least about 85%, at least about 90%, at least about 95%, at least about 96%, at least about 97%, at least about 98%, at least about 99%. or about 100% sequence identity to the amino acid sequence set forth in SEQ ID NO: 1, wherein the polypeptide comprises the amino acid sequence set forth in SEQ ID NO: 8. In some aspects, the polypeptide comprises an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1; wherein the polypeptide comprises the amino acid sequence set forth in SEQ ID NO: 8; and wherein the polypeptide comprises a Ser at the amino acid residue corresponding to amino acid 278 in SEQ ID NO: 3, a Glu at the amino acid residue corresponding to amino acid 78 in SEQ ID NO: 3, and an Asp at the amino acid residue corresponding to amino acid 82 in SEQ ID NO: 3.

In some aspects, the polypeptide comprises a deletion of one or more amino acids from the N-terminus or the C-terminus relative to the amino acid sequence set forth in SEQ ID NO: 1 or 6. In some aspects, the polypeptide comprises a deletion of at least one amino acid from the N-terminus relative to the amino acid sequence set forth in SEQ ID NO: 1 or 6. In some aspects, the polypeptide comprises a deletion of at least two amino acids from the N-terminus relative to the amino acid sequence set forth in SEQ ID NO: 1 or 6. In some aspects. the polypeptide comprises a deletion of at least three amino acids from the N-terminus relative to the amino acid sequence set forth in SEQ ID NO: 1 or 6. In some aspects, the polypeptide comprises a deletion of at least four amino acids from the N-terminus relative to the amino acid sequence set forth in SEQ ID NO: 1 or 6. In some aspects, the polypeptide comprises a deletion of at least five amino acids from the N-terminus relative to the amino acid sequence set forth in SEQ ID NO: 1 or 6. In some aspects, the polypeptide comprises a deletion of at least one amino acid from the C-terminus relative to the amino acid sequence set forth in SEQ ID NO: 1 or 6. In some aspects, the polypeptide comprises a deletion of at least two amino acids from the C-terminus relative to the amino acid sequence set forth in SEQ ID NO: 1 or 6. In some aspects, the polypeptide comprises a deletion of at least three amino acids from the C-terminus relative to the amino acid sequence set forth in SEQ ID NO: 1 or 6. In some aspects, the polypeptide comprises a deletion of at least four amino acids from the C-terminus relative to the amino acid sequence set forth in SEQ ID NO: 1 or 6. In some aspects, the polypeptide comprises a deletion of at least five amino acids from the C-terminus relative to the amino acid sequence set forth in SEQ ID NO: 1 or 6.

In some aspects, the polypeptide comprises a deletion of one or more amino acids from the N-terminus or the C-terminus relative to the amino acid sequence set forth in SEQ ID NO: 3 or 8. In some aspects, the polypeptide comprises a deletion of at least one amino acid from the N-terminus relative to the amino acid sequence set forth in SEQ ID NO: 3 or 8. In some aspects, the polypeptide comprises a deletion of at least two amino acids from the N-terminus relative to the amino acid sequence set forth in SEQ ID NO: 3 or 8. In some aspects. the polypeptide comprises a deletion of at least three amino acids from the N-terminus relative to the amino acid sequence set forth in SEQ ID NO: 3 or 8. In some aspects, the polypeptide comprises a deletion of at least four amino acids from the N-terminus relative to the amino acid sequence set forth in SEQ ID NO: 3 or 8. In some aspects, the polypeptide comprises a deletion of at least five amino acids from the N-terminus relative to the amino acid sequence set forth in SEQ ID NO: 3 or 8. In some aspects, the polypeptide comprises a deletion of at least one amino acid from the C-terminus relative to the amino acid sequence set forth in SEQ ID NO: 3 or 8. In some aspects, the polypeptide comprises a deletion of at least two amino acids from the C-terminus relative to the amino acid sequence set forth in SEQ ID NO: 3 or 8. In some aspects, the polypeptide comprises a deletion of at least three amino acids from the C-terminus relative to the amino acid sequence set forth in SEQ ID NO: 3 or 8. In some aspects, the polypeptide comprises a deletion of at least four amino acids from the C-terminus relative to the amino acid sequence set forth in SEQ ID NO: 3 or 8. In some aspects, the polypeptide comprises a deletion of at least five amino acids from the C-terminus relative to the amino acid sequence set forth in SEQ ID NO: 3 or 8.

As disclosed in the examples that follow, polypeptides according to some aspects of the disclosure are improved polypeptides for use, for example, in treating celiac sprue. The polypeptides are variants of either the processed (i.e., mature) polypeotide or the preprocessed (i.e., full-length) polypeptide corresponding to SEQ ID NO: 4 (KUMAMAX™, hereinafter referred to as Kuma010; see WO2013/023151, which is incorporated by reference herein in its entirety). Polypeptides for treating celiac sprue are capable of degrading proline (P)- and glutamine (Q)-rich components of gluten known as “gliadins” believed responsible for the bulk of the immune response in most celiac sprue patients. The polypeptides of the present disclosure show superior activity in degrading peptides having a PQLP (SEQ ID NO: 9) or PQQP (SEQ ID NO: 10) motif (such as PFPQPQLPY (SEQ ID NO: 11) and/or PFPQPQQPF (SEQ ID NO: 12)), which are substrates representative of gliadin) at pH 4 compared to Kuma011 and other polypeptides disclosed as useful for treating celiac sprue (see, e.g., WO2015/023728 and WO2016/200880, each of which are incorporated by reference herein in its entirety), and/or are shown to improve production of the polypeptides. Thus, the polypeptides of the disclosure constitute significantly improved therapeutics for treating celiac sprue.

In some aspects, the polypeptides disclosed herein are capable of degrading at pH 4 a peptide comprising an amino acid sequence selected from PFPQPQLPY (SEQ ID NO: 11), PFPQPQQPF (SEQ ID NO: 12), LQLQPFPQPQLPYPQPQLPYPQPQLPYPQPQPF (SEQ ID NO: 13), and/or FLQPQQPFPQQPQQPYPQQPQQPFPQ (SEQ ID NO: 14).

Polypeptides of the first aspect of the disclosure comprise preprocessed versions of the polypeptide enzymes of the disclosure.

Polypeptides of the first aspect of the disclosure comprise processed versions of the polypeptide enzymes of the disclosure, and also degrade a PFPQPQLPY (SEQ ID NO: 11) peptide and/or a PFPQPQQPF (SEQ ID NO: 12) peptide at pH 4, as well as LQLQPFPQPQLPYPQPQLPYPQPQLPYPQPQPF (SEQ ID NO: 13) and/or FLQPQQPFPQQPQQPYPQQPQQPFPQ (SEQ ID NO: 14).

As used herein. “at least 75% identical” or “having at least 75% sequence identity” means that the polypeptide differs in its full length amino acid sequence by 25% or less (including any amino acid substitutions, deletions, additions, or insertions) relative to a reference sequence, e.g., relative to an amino acid sequence selected from SEQ ID NOs: 1-8. In some aspects, the polypeptide comprises or consists of an amino acid sequence having at least 76%, 77%, 78%, 79%, 80%, 81%, 82%, 83%, 84%, 85%, 86%, 87%, 88%, 89%, 90%, 91%, 92%, 92%, 93%, 94%, 95%, 96%, 97%, 98%, or 99% identical to an amino acid sequence according to SEQ ID NO: 1 (preprocessed) or SEQ ID NO:8 (processed).

The polypeptide of any aspect of the polypeptides of the disclosure may comprise an amino acid substitution from SEQ ID NO: 1 or SEQ ID NO:8 at 2, 3, 4, 5, 6, 7, 8, 9, 10, 11, 12, 13, 14, 15, 16, 17, 18, 19, 20, 21, 22, 23, or all 24 (depending on the aspect) of the recited residues.

In one aspect of the polypeptides of the first aspect of the disclosure, the polypeptide comprises one or more amino acid substitutions from SEQ ID NO: 6 at one or more residues selected from the group consisting of 221D/N/Q/H, 262E, 268S/T/A, 269L/T, 270A/T/V, 319A, 354EQRY, 358S/Q/T, 368F/Q, 399Q, 402S/Q, 406S, 424K, 449E/N/Q, 461R, and 463ALMQRTV. As used throughout, the number indicates the residue number in the SEQ ID NO: 6 or SEQ ID NO: 3 polypeptide sequence, and the single letter amino acid abbreviations to the right of the number indicate the possible amino acid substitutions compared to the amino acid residue present at that position in SEQ ID NO: 6 or 3.

In another aspect of the polypeptides of the first aspect of the disclosure, the polypeptide comprises amino acid substitutions from SEQ ID NO: 6 at residues 399 and 449. In one aspect, the polypeptide comprises amino acid substitutions 399Q and 449Q. In some aspects, the polypeptide comprises a Q at position 399 and a Q at position 449, based on the numbering of SEQ ID NO: 6.

In a further aspect of the polypeptides of the first aspect of the disclosure, the polypeptide comprises 358S and 463T. In some aspects, the polypeptide comprises (i) an S at position 358, and (ii) a T at position 463, or any combination of (i)-(ii), based on the numbering of SEQ ID NO: 6.

In one aspect of the polypeptides of the first aspect of the disclosure, the polypeptide comprises 262E, 269T, 354Q, 358S, 399Q, 449Q, and 463T. In some aspects, the polypeptide comprises (i) an E at position 262, (ii) a T at position 269, (iii) a Q at position 354, (iv) an S at position 358, (v) a Q at position 399, (vi) a Q at position 449, and (vii) a T at position 463, or any combination of (i)-(vii), based on the numbering of SEQ ID NO: 6. These polypeptide are extensively characterized in the examples disclosed in in WO2016/200880, as exemplified by the polypeptide designated as Kuma030 and variants thereof. In another aspect of the polypeptides of the first aspect of the disclosure, the polypeptide comprises 319A, 368F, 399Q, 449Q, and I463T. In some aspects, the polypeptide comprises (i) an A at position 319, (ii) an F at position 368, (iii) a Q at position 399, (iv) a Q at position 449, and a (v) T at position 463, or any combination of (i)-(v), based on the numbering of SEQ ID NO: 6. These polypeptide are extensively characterized in the examples disclosed in in WO2016/200880, as exemplified by the polypeptide designated as Kuma040 and variants thereof. In a further aspect of the polypeptides of the first aspect of the disclosure, the polypeptide comprises 262E, 269T, 270V, 354Q, 358S, 399Q, and A449Q. In some aspects, the polypeptide comprises (i) an E at position 262, (ii) a T at position 269, (iii) a V at position 270, (vi) a Q at position 354, (v) an S at position 358, (vi) a Q at position 399, and (vii) a Q at position 449, or any combination of (i)-(vii), based on the numbering of SEQ ID NO: 6. These polypeptide are extensively characterized in the examples disclosed in in WO2016/200880, as exemplified by the polypeptide designated as Kuma050 and variants thereof. In one aspect of the polypeptides of the first aspect of the disclosure, the polypeptide comprises 262E, 269T, 320M, 354Q, 358S, 399Q, 449Q, and 463T. In some aspects, the polypeptide comprises (i) an E at position 262, (ii) a T at position 269, (iii) a M at position 320, (vi) a Q at position 354, (v) an S at position 358, (vi) a Q at position 399, and (vii) a Q at position 449, or any combination of (i)-(vii), based on the numbering of SEQ ID NO: 6. These polypeptide are extensively characterized in the examples disclosed in in WO2016/200880, as exemplified by the polypeptide designated as Kuma060 and variants thereof. In a still further aspect of the polypeptides of the first aspect of the disclosure, the polypeptide comprises, 319A, 320M, 368F, 399Q, 449Q, and 463T. In some aspects, the polypeptide comprises (i) an A at position 319 (ii) an M at position 320, (iii) an F at position 368. (v) a Q at position 399, and (v) a Q at position 449. or any combination of (i)-(v). based on the numbering of SEQ ID NO: 6. These polypeptide are extensively characterized in the examples disclosed in in WO2016/200880, as exemplified by the polypeptide designated as Kuma070 and variants thereof. As used herein, the terms “Kuma020,” “Kuma030,” “Kuma040,” “Kuma050,” and “Kuma070” refer to the same polypeptides with the same designation as disclosed in WO2016/200880.

In another aspect of the polypeptides of the first aspect of the disclosure, the polypeptides comprise an amino acid substitution from SEQ ID NO: 6 at one or more amino acid positions selected from the group consisting of 105, 171, 172, 173, 174, and 456. In one aspect, the amino acid substitution is 105H; 171R A, or S; 172R, A, or S; 173R or S, 174S, and/or 456V. In some aspects, the polypeptide comprises (i) an H at position 105; (ii) an R, A, or S at position 171; (iii) an R, A, or S at position 172; (iv) and R or S at position 173; (v) an S a position 174; (vi) a V at position 456; or (vii) any combination of (i)-(vi), based on the numbering of SEQ ID NO: 6. In another aspect, the amino acid substitution is 171R, 172R, and/or 456V. In some aspects, the polypeptide comprises (i) an R at position 171, (ii) an R at position 172, (iii) a V at position 456, or (iv) any combination of (i)-(iii), based on the numbering of SEQ ID NO: 6.

In one aspect of the polypeptides of the second aspect of the disclosure the polypeptide comprises one or more amino acid substitution from SEQ ID NO: 3 at one or more residues selected from the group consisting of 32D/N/Q/H, 73E, 79S/T/A, 80L/T, 81A/T/V, 130A, 165E/Q/R/Y, 169S/Q/T, 179F/Q, 210Q, 213S/Q, 217S, 235K, 260E/N/Q, 272R, and 274A/L/M/Q/R/T/V. In another aspect of the polypeptides of the second aspect of the disclosure, the polypeptide comprises amino acid substitutions from SEQ ID NO: 3 at residues 210 and 260. In a further aspect of the polypeptides of the second aspect of the disclosure, the polypeptide comprises amino acid substitutions 210Q and 260Q. In some aspects, the polypeptide comprises (i) a Q at position 210, (ii) an Q at position 260, or any combination of (i)-(ii), based on the numbering of SEQ ID NO: 3. In one aspect of the polypeptides of the second aspect of the disclosure, the polypeptide comprises 169S and 274T. (Kuma020 genus). In such an aspect, the polypeptide comprises (i) an S at position 169, (ii) a T at position 274, or (iv) any combination of (i)-(ii), based on the numbering of SEQ ID NO: 3. In another aspect of the polypeptides of the second aspect of the disclosure the polypeptide comprises 73E, 80T, 165Q, 169S, 210Q, 260Q, and 274T. (Kuma030 genus). In such an aspect, the polypeptide comprises (i) an E at position 73, (ii) a T at position 80, (iii) a Q at position 165, (iv) an S at position 169, (v) a Q at position 210, (vi) a Q at position 260, and (vii) a T at position 274, or any combination of (i)-(vii), based on the numbering of SEQ ID NO: 3. In a further aspect of the polypeptides of the second aspect of the disclosure, the polypeptide comprises 130A, 179F, 210Q, 260Q, and 274T. (Kuma040 genus). In such an aspect, the polypeptide comprises (i) an A at position 130, (ii) an F at position 179, (iii) a Q at position 210, (iv) a Q at position 260, (v) a T at position 274, or any combination of (i)-(v), based on the numbering of SEQ ID NO: 3. In a still further aspect of the polypeptides of the second aspect of the disclosure, the polypeptide comprises 73E, 80T, 81V, 165Q, 169S, 210Q, and 260Q. (Kuma050 genus). In such an aspect, the polypeptide comprises (i) an E at position 73, (ii) a T at position 80, (iii) a V at position 81, (iv) a Q at position 165, (v) an S at position 169, (vi) a Q at position 210 (vii) a Q at position 260, or any combination of (i)-(vii), based on the numbering of SEQ ID NO: 3. In one aspect of the polypeptides of the second aspect of the disclosure, the polypeptide comprises 73E, 80T, 320M, 165Q, 169S, 210Q, 260Q, and 274T. (Kuma060 genus). In such an aspect, the polypeptide comprises (i) an E at position 73, (ii) a T at position 80, (iii) an M at position 320, (iv) a Q at position 165, (v) an S at position 169, (vi) a Q at position 210 (vii) a Q at position 260, (viii) a T at position 274, or any combination of (i)-(vii), based on the numbering of SEQ ID NO: 3. In another aspect of the polypeptides of the second aspect of the disclosure, the polypeptide comprises 130A, 131M, 179F, 210Q, 260Q, and 274T. (Kuma070 genus). In such an aspect, the polypeptide comprises (i) an A at position 130, (ii) an M at position 131, (iii) an F at position 179, (iv) a Q at position 210, (v) a Q at position 260, (vi) a T at position 274, or any combination of (i)-(vi), based on the numbering of SEQ ID NO: 3. In a still further aspect of the polypeptides of the second aspect of the disclosure, the polypeptides comprise an amino acid substitution from SEQ ID NO: 3 at one or more amino acid positions selected from the group consisting of 267. In one aspect, the amino acid substitution is 267V. In such an aspect, the polypeptide comprises a V at position 267, based on the numbering of SEQ ID NO: 3.

In a further aspect of the polypeptides of any aspect of the disclosure, the polypeptides further comprise a histidine tag at the C-terminus of the polypeptide, to facilitate isolation of the polypeptide. Any suitable histidine tag can be used: in one aspect the tag is linked to a TEV protease cut site (ENLYFQS) (SEQ ID NO: 18) to allow for its efficient removal with TEV protease after purification, for example, the tag may comprise or consist of the amino acid sequence GSTENLYFQSGALEHHHHHH (SEQ ID NO: 17). In another aspect, the histidine tag is a cleavable histidine tag, permitting easier removal of the His-tag. In one aspect, the cleavable histidine tag comprises the amino acid sequence X_{N}PQ(L/Q)PX_{N}HHHHHH (SEQ ID NO: 15), wherein Xx is an linker of between 1-25 amino acid residues. In one non-limiting example, the cleavable histidine tag comprises the amino acid sequence GSSGSSGSQPQLPYGSSGSSGSHHHHHH (SEQ ID NO: 16).

In one aspect of any aspect of the polypeptides of the disclosure, amino acid substitutions compared to SEQ ID NO: 6 or SEQ ID NO: 3 may comprise one or more of the substitutions noted in Tables 2 or 3. Substitutions at these positions were found to be generally well-tolerated (i.e. generally result in minor to no effects on activity), and in some cases to increase the activity of the polypeptides of the disclosure by no more than 20%.

TABLE 2
Possible Amino Acid Substitutions at Position Relative to Kuma010.

| Residue number | | Residue number | |
|---|---|---|---|
| (preprocessed/processed) | Residue | (preprocessed/processed) | Residue |
| 221/32 | D, N, Q, H | 358/169 | A, S, N, Q, T |
| 261/72 | A, R, N, D, C, Q, E, G, | 368/179 | A, R, N, C, Q, E, G, K, |
| | H, I, L, K, M, S, T, W, Y, | | M, F, S, T, W, Y |
| | V | | |
| 262/73 | A, R, N, D, C, Q, E, G, | 397/208 | A, C, F, Y |
| | H, I, L, M, F, T, W, Y, V | | |
| 264/75 | A, N, D, C, Q, E, G, S, T, | 399/210 | Q, N |
| | Y | | |
| 266/77 | A, C, S | 402/213 | Q, N, S |
| 268/79 | S, T | 406/217 | S |
| 269/80 | L, T | 424/235 | K |
| 270/81 | A, R, N, D, C, Q, E, G, I, | 446/257 | G, S |
| | K, S, T, V | | |
| 317/128 | A, N, C, G, T, V | 448/259 | A, R, N, D, C, Q, E, G, |
| | | | H, I, L, K, M, F, S, T, W, |
| | | | Y, V |
| 318/129 | A, R, N, D, C, Q, E, G, | 449/260 | Q, E, G, N |
| | H, L, K, M, F, S, T, Y, V | | |
| 319/130 | A, N, D, C, Q, H, M, T | 456/267 | A, N, D, C, Q, E, G, H, |
| | | | L, S, T, V |
| 320/131 | A, R, N, D, C, Q, K, M, S | 461/272 | R |
| 350/161 | N, D, C, G, S, T | 463/274 | A, R, N, D, C, Q, E, G, |
| | | | H, L, K, M, F, S, T, W, |
| | | | Y, V |
| 351/162 | G, S | 464/277 | A, N, D, C, S, |
| 353/164 | A, R, N, C, Q, E, G, I, K, | 466/279 | D, C, G, S |
| | M, S, T, V | | |
| 354/165 | A, R, N, D, C, Q, E, G, | | |
| | H, L, K, M, F, T, W, Y | | |

In another embodiment of any aspect of the polypeptides of the disclosure, amino acid substitutions compared to SEQ ID NO: 6 or SEQ ID NO: 3 may comprise one or more of the substitutions noted in Table 3.

TABLE 3

| Residue number | | Residue number | |
|---|---|---|---|
| (preprocessed/processed) | Residue | (preprocessed/processed) | Residue |
| 221/32 | D, N, Q, H | 358/169 | A, S, N, Q, T |
| 261/72 | S | 368/179 | A, N, D, Q, E, S, T |
| 262/73 | A, R, N, D, Q, E, G, L, | 402/213 | Q, S |
| | M, T | | |
| 264/75 | A | 406/217 | S |
| 268/79 | S, T | 424/235 | K |
| 269/80 | L, T | 446/257 | S |
| 270/81 | A, T, V | 449/260 | Q, N, A |
| 317/128 | A, T | 456/267 | V |
| 319/130 | A | 461/272 | R |
| 354/165 | A, R, N, D, Q, E, K, T, | 463/274 | A, R, Q, L, M, T, V |
| | Y | | |

In another embodiment of any aspect of the polypeptides of the disclosure, amino acid at each residue of the polypeptides of the disclosure may be as noted in Table 4, which lists all of the possible mutations at each position in the polypeptide enzymes as predicted by computational mutagenesis analysis. As described in the examples disclosed in in WO2016/200880, mutations were tested at each position found in the active site (residues 261-264, 266-267, 270, 317-320, 350-354, 368, 397, 403-404, 446, 448, 456, and 463-468) using degenerate primers to test the effects of various amino acid substitutions on activity; those that did not interfere with activity can be incorporated in the polypeptides of the disclosure, as reflected in Table 4.

TABLE 4
Possible Amino Acids at Residues Relative to Kuma 010

| Full | | |
|---|---|---|
| Length | Mature | Amino Acid Possibilities |
| 190 | 1 | ALA, ARG, ASN, ASP, CYS, GLN, GLU, GLY, HIS, ILE, LEU, LYS, MET, PHE, PRO, SER, THR, TRP, TYR, |
| | | VAL |
| 191 | 2 | ALA, ARG, ASN, ASP, CYS, GLN, GLU, GLY, HIS, ILE, LYS, MET, PHE, PRO, SER, THR, TRP, VAL |
| 192 | 3 | ALA, ARG, ASN, ASP, CYS, GLN, GLU, GLY, HIS, LEU, LYS, MET, PHE, PRO, SER, TRP, TYR |
| 193 | 4 | ALA, ARG, ASN, ASP, CYS, GLN, GLU, GLY, HIS, ILE, LEU, LYS, MET, PHE, PRO, SER, THR, TRP, TYR, |
| | | VAL |
| 194 | 5 | ALA, ARG, ASN, ASP, CYS, GLN, GLU, GLY, HIS, LEU, LYS, MET, PHE, SER, THR, TRP, TYR, VAL |
| 195 | 6 | ALA, ASN, CYS, GLN, HIS, LEU, MET, PHE, THR, TYR |
| 196 | 7 | ALA, ARG, ASN, ASP, CYS, GLN, GLU, GLY, HIS, LYS, MET, PHE, SER, THR, TRP, TYR |
| 197 | 8 | ALA, GLY, PRO, SER |
| 198 | 9 | ALA, ARG, ASN, ASP, CYS, GLN, GLU, GLY, HIS, ILE, LEU, LYS, MET, PHE, SER, THR, TRP, TYR, VAL |
| 199 | 10 | ALA, ARG, ASN, ASP, CYS, GLN, GLU, GLY, HIS, LEU, LYS, MET, PHE, SER, THR, TRP, TYR |
| 200 | 11 | ALA, ASN, ASP, CYS, GLY, ILE, SER, THR, VAL |
| 201 | 12 | ALA, CYS, GLY, SER |
| 202 | 13 | ALA, ARG, ASN, ASP, CYS, GLN, GLU, GLY, HIS, ILE, LEU, LYS, MET, PHE, SER, THR, TRP, TYR, VAL |
| 203 | 14 | ALA, GLY, SER |
| 204 | 15 | ALA, ASN, ASP, CYS, GLN, GLU, GLY, HIS, ILE, LEU, LYS, MET, PHE, SER, THR, TYR |
| 205 | 16 | ALA, ARG, ASN, ASP, CYS, GLN, GLU, GLY, HIS, ILE, LEU, LYS, MET, PHE, SER, THR, TRP, TYR, VAL |
| 206 | 17 | ALA, ASN, ASP, CYS, GLN, GLU, GLY, HIS, ILE, LEU, LYS, MET, PHE, SER, THR, TYR, VAL |
| 207 | 18 | ALA, CYS, GLN, GLU, GLY, LYS, PRO, SER, THR, TRP |
| 208 | 19 | ALA, ARG, ASN, ASP, CYS, GLN, GLU, GLY, HIS, ILE, LEU, LYS, MET, PHE, PRO, SER, THR, TRP, TYR, |
| | | VAL |
| 209 | 20 | ALA, ARG, ASN, ASP, CYS, GLN, GLU, GLY, HIS, ILE, LEU, LYS, MET, PHE, SER, THR, TRP, TYR, VAL |
| 210 | 21 | ALA, ARG, ASN, ASP, CYS, GLN, GLU, GLY, LEU, MET, SER, THR, VAL |
| 211 | 22 | ALA, ARG, ASN, ASP, CYS, GLN, GLU, GLY, HIS, LYS, MET, PHE, SER, THR, TYR |
| 212 | 23 | GLY |
| 213 | 24 | ALA, ARG, ASN, ASP, CYS, GLN, GLU, GLY, HIS, LEU, LYS, MET, PHE, SER, THR, TRP, TYR, VAL |
| 214 | 25 | GLY |
| 215 | 26 | ALA, ASN, ASP, CYS, GLN, GLU, GLY, SER, THR |
| 216 | 27 | ALA, ASN, ASP, CYS, GLN, GLY, SER, THR, VAL |
| 217 | 28 | ALA, CYS, ILE, LEU, SER, THR, VAL |
| 218 | 29 | ALA, GLY, SER |
| 219 | 30 | ALA, ASN, ASP, CYS, GLN, GLU, GLY, HIS, ILE, LEU, MET, SER, THR, VAL |
| 220 | 31 | ALA, ASN, ASP, CYS, GLN, GLU, GLY, ILE, SER, THR, VAL |
| 221 | 32 | ALA, ASN, ASP, CYS, GLN, GLU, GLY, ILE, SER, THR, VAL |
| 222 | 33 | ALA, ASN, ASP, CYS, GLN, GLU, GLY, ILE, LEU, LYS, SER, THR, VAL |
| 223 | 34 | ALA, ARG, ASN, ASP, CYS, GLU, GLY, LYS, MET, SER |
| 224 | 35 | GLY |
| 225 | 36 | GLY |
| 226 | 37 | ALA, ARG, ASN, ASP, CYS, GLU, GLY, HIS, LEU, PHE, SER, THR, TRP, TYR |
| 227 | 38 | ALA, ARG, ASN, ASP, CYS, GLN, GLU, GLY, LYS, MET, SER |
| 228 | 39 | ALA, ARG, ASN, ASP, CYS, GLN, GLU, GLY, HIS, ILE, LEU, LYS, MET, PHE, PRO, SER, THR, TRP, TYR, |
| | | VAL |
| 229 | 40 | ALA, ARG, ASN, ASP, CYS, GLN, GLU, GLY, HIS, ILE, LEU, LYS, MET, PHE, SER, THR, TRP, TYR, VAL, |
| 230 | 41 | ALA, GLY, SER |
| 231 | 42 | ALA, ASN, ASP, CYS, GLN, GLU, GLY, LEU, SER, THR |
| 232 | 43 | ALA, ARG, ASN, ASP, CYS, GLN, GLU, GLY, HIS, ILE, LEU, LYS, MET, PHE, SER, THR, TRP, TYR, VAL, |
| 233 | 44 | ALA, ARG, ASN, ASP, CYS, GLN, GLU, GLY, HIS, ILE, LEU, LYS, MET, PHE, SER, THR, TRP, TYR, VAL, |
| 234 | 45 | ALA, ASN, CYS, GLY, HIS, PHE, SER, TYR |
| 235 | 46 | ALA, ASN, ASP, CYS, HIS, MET, PHE, SER, THR, TRP, TYR |
| 236 | 47 | ALA, ARG, ASN, ASP, CYS, GLN, GLU, GLY, HIS, ILE, LEU, LYS, MET, PHE, SER, THR, TRP, TYR, VAL, |
| 237 | 48 | ALA, ARG, ASN, ASP, CYS, GLN, GLU, GLY, HIS, ILE, LEU, LYS, MET, PHE, SER, THR, TRP, TYR, VAL, |
| 238 | 49 | ALA, ARG, ASN, ASP, CYS, GLN, GLU, GLY, HIS, ILE, LEU, MET, SER, THR, VAL |
| 239 | 50 | GLY |
| 240 | 51 | ALA, ARG, ASN, ASP, CYS, GLN, GLU, GLY, HIS, LEU, LYS, MET, SER, THR, TYR, VAL |
| 241 | 52 | ALA, ARG, ASN, ASP, CYS, GLN, GLU, GLY, HIS, ILE, LEU, LYS, MET, PHE, PRO, SER, THR, TRP, TYR, |
| | | VAL |
| 242 | 53 | ALA, ARG, ASN, ASP, CYS, GLN, GLU, GLY, HIS, ILE, LYS, MET, PRO, SER, THR, VAL |
| 243 | 54 | ALA, GLY, PRO, SER |
| 244 | 55 | ALA, ARG, ASN, ASP, CYS, GLN, GLU, GLY, HIS, ILE, LEU, LYS, MET, PHE, PRO, SER, THR, TRP, TYR, |
| | | VAL |
| 245 | 56 | ALA, ASN, CYS, GLY, SER, THR, VAL |
| 246 | 57 | ALA, ARG, ASN, ASP, CYS, GLN, GLU, GLY, HIS, ILE, LEU, LYS, MET, PHE, SER, THR, TRP, TYR, VAL |
| 247 | 58 | ALA, ARG, ASP, CYS, GLY, ILE, LYS, MET, PRO, SER |
| 248 | 59 | ALA, ARG, ASN, ASP, CYS, GLN, GLU, GLY, HIS, ILE, LYS, MET, PHE, SER, THR, TRP, TYR, VAL |
| 249 | 60 | ALA, ARG, ASN, ASP, CYS, GLN, GLU, GLY, ILE, LEU, LYS, MET, PRO, SER, THR |
| 250 | 61 | ALA, ASN, ASP, CYS, GLN, GLU, GLY, ILE, SER, THR, VAL |
| 251 | 62 | ALA, ARG, ASN, ASP, CYS, GLN, GLU, GLY, HIS, ILE, LEU, LYS, MET, PHE, SER, THR, TYR, VAL |
| 252 | 63 | ASN, ASP, GLY, SER |
| 253 | 64 | ALA, ARG, ASN, ASP, CYS, GLN, GLU, GLY, HIS, LYS, MET, PHE, SER, THR, TRP |
| 254 | 65 | ALA, ARG, ASN, ASP, CYS, GLN, GLU, GLY, HIS, ILE, LEU, LYS, MET, PHE, SER, THR, TRP, TYR, VAL |
| 255 | 66 | ALA, ARG, ASN, ASP, CYS, MET, SER, THR |
| 256 | 67 | ALA, ARG, ASN, ASP, CYS, GLN, GLU, GLY, HIS, ILE, LEU, LYS, MET, PHE, SER, THR, TRP, TYR, VAL |
| 257 | 68 | ALA, ARG, ASN, CYS, GLN, GLU, GLY, ILE, LYS, MET, PRO, SER, THR, VAL |
| 258 | 69 | ALA, ARG, ASN, ASP, CYS, GLN, GLU, GLY, HIS, ILE, LEU, LYS, MET, PHE, SER, THR, TRP, TYR, VAL |
| 259 | 70 | GLY |
| 260 | 71 | ALA, ARG, ASN, ASP, CYS, GLN, GLU, GLY, HIS, ILE, LEU, LYS, MET, PHE, SER, THR, TRP, TYR, VAL |
| 261 | 72 | ALA, ARG, ASN, ASP, CYS, GLN, GLU, GLY, HIS, ILE, LEU, LYS, MET, PRO, SER, THR, TRP, TYR, VAL |
| 262 | 73 | ALA, ARG, ASN, ASP, CYS, GLN, GLU, GLY, HIS, ILE, LEU, LYS, MET, PHE, SER, THR, TRP, TYR, VAL |
| 263 | 74 | GLY |
| 264 | 75 | ALA, ASN, ASP, CYS, GLN, GLU, GLY, PRO, SER, THR, TRP |
| 265 | 76 | ALA, ASN, ASP, CYS, GLN, GLU, GLY, SER, THR, VAL |
| 266 | 77 | ALA, CYS, GLY, SER |
| 267 | 78 | GLU |
| 268 | 79 | ALA, ASN, ASP, CYS, GLY, SER, THR, VAL |
| 269 | 80 | ALA, ARG, ASN, ASP, CYS, GLN, GLU, GLY, ILE, LEU, LYS, MET, SER, THR, VAL |
| 270 | 81 | ALA, ARG, ASN, ASP, CYS, GLN, GLU, GLY, ILE, LEU, LYS, SER, THR, VAL |
| 271 | 82 | ASP |
| 272 | 83 | ALA, ASN, ASP, CYS, GLN, GLU, GLY, ILE, MET, SER, THR, VAL |
| 273 | 84 | ALA, ASN, ASP, CYS, GLN, GLU, GLY, SER, THR |
| 274 | 85 | ALA, ASN, ASP, CYS, GLY, ILE, SER, THR, VAL |
| 275 | 86 | ALA, CYS, GLY, SER |
| 276 | 87 | GLY |
| 277 | 88 | ALA, GLY, SER |
| 278 | 89 | ALA, ASN, ASP, CYS, GLN, GLU, GLY, ILE, LEU, MET, SER, THR, VAL |
| 279 | 90 | ALA, GLY, SER |
| 280 | 91 | ALA, ASN, ASP, CYS, GLN, GLU, GLY, HIS, MET, PHE, PRO, SER, TRP, TYR |
| 281 | 92 | GLY |
| 282 | 93 | ALA, GLY, SER |
| 283 | 94 | ALA, ARG, ASN, ASP, CYS, GLN, GLU, GLY, HIS, ILE, LEU, LYS, MET, PHE, SER, THR, TRP, TYR, VAL |
| 284 | 95 | CYS, HIS, ILE, LEU, MET, PHE, THR, TYR, VAL |
| 285 | 96 | ALA, GLY, SER |
| 286 | 97 | ALA, ASN, ASP, CYS, GLY, SER, THR, VAL |
| 287 | 98 | ALA, ASN, ASP, CYS, GLN, HIS, LEU, PHE, SER, TYR |
| 288 | 99 | HIS, PHE |
| 289 | 100 | ALA, GLY, SER |
| 290 | 101 | ALA, ARG, ASN, ASP, CYS, GLN, GLU, GLY, HIS, ILE, LEU, LYS, MET, PHE, PRO, SER, THR, TRP, TYR, |
| | | VAL |
| 291 | 102 | ALA, ARG, ASN, ASP, CYS, GLN, GLU, GLY, HIS, LEU, LYS, MET, PHE, PRO, SER, THR, TRP, TYR, VAL, |
| 292 | 103 | ALA, ARG, ASN, ASP, CYS, GLN, GLU, GLY, HIS, LEU, LYS, MET, PHE, SER, THR, TRP, TYR, VAL |
| 293 | 104 | ALA, ARG, ASN, ASP, CYS, GLN, GLU, GLY, HIS, ILE, LEU, LYS, MET, PRO, SER, THR, VAL |
| 294 | 105 | ALA, ARG, ASN, ASP, CYS, GLN, GLU, GLY, HIS, ILE, LEU, LYS, MET, PHE, PRO, SER, THR, TRP, TYR, |
| | | VAL |
| 295 | 106 | GLY |
| 296 | 107 | ALA, ASN, ASP, CYS, GLN, GLU, GLY, HIS, LEU, LYS, MET, PHE, SER, THR, VAL |
| 297 | 108 | ALA, ARG, ASN, ASP, CYS, GLN, GLU, GLY, HIS, LEU, LYS, MET, PHE, SER, THR, TRP, TYR |
| 298 | 109 | ALA, ARG, ASN, ASP, CYS, GLN, GLU, GLY, HIS, ILE, LEU, LYS, MET, PHE, SER, THR, TRP, TYR, VAL, |
| 299 | 110 | ALA, GLY, SER |
| 300 | 111 | ALA, ASN, ASP, CYS, GLN, GLU, GLY, ILE, LEU, LYS, MET, SER, THR, VAL |
| 301 | 112 | ALA, ARG, ASN, ASP, CYS, GLN, GLU, GLY, ILE, LEU, LYS, MET, PHE, SER, THR, VAL |
| 302 | 113 | ALA, ARG, ASN, ASP, CYS, GLN, GLU, GLY, HIS, LYS, MET, SER, THR, TRP, VAL |
| 303 | 114 | ALA, GLY, SER |
| 304 | 115 | ALA, ASN, ASP, CYS, GLN, GLU, GLY, ILE, LEU, SER, THR, VAL |
| 305 | 116 | ALA, ARG, ASN, ASP, CYS, GLN, GLU, GLY, HIS, LEU, LYS, MET, PHE, SER, THR, TRP, TYR, VAL |
| 306 | 117 | ALA, ASN, ASP, SER |
| 307 | 118 | ALA, ARG, ASN, ASP, CYS, GLN, GLU, GLY, HIS, ILE, LEU, LYS, MET, PHE, PRO, SER, THR, TRP, TYR, |
| | | VAL |
| 308 | 119 | ALA, ARG, ASN, ASP, CYS, GLN, GLU, GLY, HIS, ILE, LEU, LYS, MET, PHE, SER, THR, TRP, TYR, VAL, |
| 309 | 120 | ALA, ARG, ASN, ASP, CYS, GLN, GLU, GLY, HIS, LEU, LYS, MET, PHE, SER, THR, TRP, TYR |
| 310 | 121 | ALA, ARG, ASN, ASP, CYS, GLN, GLU, GLY, HIS, ILE, LEU, LYS, MET, PHE, SER, THR, TRP, TYR, VAL, |
| 311 | 122 | ALA, CYS, GLY, PRO, SER |
| 312 | 123 | ALA, ARG, ASN, ASP, CYS, GLN, GLU, GLY, HIS, LYS, MET, PHE, SER, THR, TRP, TYR |
| 313 | 124 | ALA, CYS, GLY, ILE, SER, THR, VAL |
| 314 | 125 | ALA, ASN, ASP, CYS, GLN, GLU, GLY, ILE, SER, THR, VAL |
| 315 | 126 | ALA, CYS, GLY, SER, THR |
| 316 | 127 | ALA, ASN, ASP, CYS, GLN, GLU, GLY, ILE, LEU, MET, SER, THR, VAL |
| 317 | 128 | ALA, ASN, CYS, GLY, SER, THR, VAL |
| 318 | 129 | ALA, ARG, ASN, ASP, CYS, GLN, GLU, GLY, HIS, LEU, LYS, MET, PHE, SER, THR, TRP, TYR, VAL |
| 319 | 130 | ALA, ASN, ASP, CYS, GLN, GLY, HIS, MET, SER, THR |
| 320 | 131 | ALA, ARG, ASN, ASP, CYS, GLN, GLY, LYS, MET, SER |
| 321 | 132 | ALA, CYS, GLY, PRO, SER |
| 322 | 133 | ALA, ASP, CYS, GLN, GLU, GLY, LEU, SER |
| 323 | 134 | ALA, ARG, ASN, ASP, CYS, GLN, GLU, GLY, HIS, ILE, LEU, LYS, MET, PHE, PRO, SER, THR, TRP, VAL, |
| 324 | 135 | ALA, ARG, ASN, ASP, CYS, GLN, GLU, GLY, HIS, ILE, LEU, LYS, MET, PHE, SER, THR, TRP, TYR, VAL, |
| 325 | 136 | ALA, ARG, ASN, ASP, CYS, GLN, GLU, GLY, HIS, LYS, MET, PHE, SER, TRP, TYR |
| 326 | 137 | ALA, ARG, ASN, ASP, CYS, GLN, GLU, GLY, HIS, LEU, LYS, MET, PHE, SER, THR, TRP, TYR |
| 327 | 138 | ALA, ARG, ASN, ASP, CYS, GLN, GLU, GLY, HIS, ILE, LEU, LYS, MET, PHE, PRO, SER, THR, TRP, TYR, |
| | | VAL |
| 328 | 139 | ALA, ARG, ASN, ASP, CYS, GLN, GLU, GLY, HIS, ILE, LEU, LYS, MET, PHE, SER, THR, TRP, TYR, VAL, |
| 329 | 140 | ALA, ASP, CYS, GLY, SER |
| 330 | 141 | ALA, ARG, ASN, ASP, CYS, GLN, GLU, GLY, ILE, LEU, LYS, MET, SER, THR, VAL |
| 331 | 142 | ALA, ARG, ASN, ASP, CYS, GLN, GLU, GLY, HIS, ILE, LEU, LYS, MET, PHE, SER, THR, TRP, TYR, VAL, |
| 332 | 143 | ALA, ARG, ASN, ASP, CYS, GLN, GLU, GLY, HIS, ILE, LEU, LYS, MET, PHE, SER, THR, TRP, TYR, VAL, |
| 333 | 144 | ALA, ASN, ASP, CYS, GLN, GLU, GLY, HIS, ILE, LEU, LYS, MET, SER, THR, VAL |
| 334 | 145 | ALA, ARG, ASN, ASP, CYS, GLU, GLY, MET, SER, THR, VAL |
| 335 | 146 | ALA, ARG, ASN, ASP, CYS, GLN, GLU, GLY, HIS, ILE, LEU, LYS, MET, PHE, SER, THR, TRP, TYR, VAL, |
| 336 | 147 | ALA, ARG, CYS, GLN, GLU, GLY, MET, SER |
| 337 | 148 | ALA, ARG, ASN, ASP, CYS, GLN, GLU, GLY, HIS, LEU, LYS, MET, PHE, SER, THR, TRP, TYR, VAL |
| 338 | 149 | ALA, ARG, ASN, ASP, CYS, GLN, GLU, GLY, HIS, ILE, LEU, LYS, MET, PHE, SER, THR, TRP, TYR, VAL, |
| 339 | 150 | ALA, ARG, ASN, ASP, CYS, GLN, GLU, GLY, HIS, ILE, LEU, LYS, MET, PHE, SER, THR, TRP, TYR, VAL, |
| 340 | 151 | ALA, ASN, ASP, GLY, SER |
| 341 | 152 | ALA, ARG, ASN, ASP, CYS, GLN, GLU, GLY, HIS, ILE, LYS, MET, SER, THR, VAL |
| 342 | 153 | ALA, ARG, ASN, ASP, CYS, GLN, GLU, GLY, HIS, ILE, LEU, LYS, MET, PHE, SER, THR, TRP, TYR, VAL, |
| 343 | 154 | ALA, ARG, ASN, ASP, CYS, GLN, GLU, GLY, HIS, ILE, LEU, LYS, MET, PHE, SER, THR, TRP, TYR, VAL, |
| 344 | 155 | ALA, ARG, ASN, ASP, CYS, GLN, GLU, GLY, HIS, LEU, LYS, MET, PHE, SER, THR, TRP, TYR |
| 345 | 156 | ALA, ARG, ASN, ASP, CYS, GLN, GLU, GLY, HIS, ILE, LEU, LYS, MET, PHE, SER, THR, TYR, VAL |
| 346 | 157 | ALA, ASN, ASP, CYS, GLN, GLU, GLY, HIS, LYS, MET, PHE, SER, THR |
| 347 | 158 | ALA, ASN, ASP, CYS, GLN, GLU, GLY, ILE, LYS, MET, PRO, SER, THR, VAL |
| 348 | 159 | ALA, ASN, ASP, CYS, GLN, GLU, GLY, LEU, SER, THR, VAL |
| 349 | 160 | ALA, CYS, GLY, SER, THR |
| 350 | 161 | ALA, ASN, ASP, CYS, GLY, SER, THR |
| 351 | 162 | ALA, GLY, SER |
| 352 | 163 | GLY |
| 353 | 164 | ALA, ARG, ASN, ASP, CYS, GLN, GLU, GLY, ILE, LYS, MET, SER, THR, VAL |
| 354 | 165 | ALA, ARG, ASN, ASP, CYS, GLN, GLU, GLY, HIS, LEU, LYS, MET, PHE, SER, THR, TRP, TYR |
| 355 | 166 | GLY |
| 356 | 167 | ALA, GLY, SER |
| 357 | 168 | ALA, ARG, ASN, ASP, CYS, GLN, GLU, GLY, ILE, MET, SER, THR, VAL |
| 358 | 169 | ALA, GLY, SER |
| 359 | 170 | ASN, GLY |
| 360 | 171 | ALA, ARG, ASN, ASP, CYS, GLN, GLU, GLY, ILE, LEU, LYS, MET, SER, THR, VAL |
| 361 | 172 | ALA, ARG, ASN, ASP, CYS, GLN, GLU, GLY, HIS, ILE, LEU, LYS, MET, PHE, SER, THR, TRP, TYR, VAL, |
| 362 | 173 | ALA, ARG, ASN, ASP, CYS, GLN, GLU, GLY, HIS, ILE, LEU, LYS, MET, PHE, SER, THR, TRP, TYR, VAL, |
| 363 | 174 | ASN, ASP, GLY, SER |
| 364 | 175 | ALA, ARG, ASN, ASP, CYS, GLN, GLU, GLY, HIS, LEU, LYS, MET, PHE, SER, THR, TRP, TYR |
| 365 | 176 | ALA, ARG, ASN, ASP, CYS, GLY, HIS, MET, PHE, SER, THR, TRP, TYR |
| 366 | 177 | ALA, ASN, ASP, CYS, HIS, LYS, SER |
| 367 | 178 | ALA, ASP, CYS, GLY, SER, THR, VAL |
| 368 | 179 | ALA, ARG, ASN, ASP, CYS, GLN, GLU, GLY, HIS, LYS, MET, PHE, SER, THR, TRP, TYR |
| 369 | 180 | ALA, CYS, HIS, PHE, SER, TYR |
| 370 | 181 | ALA, ASP, CYS, GLY, PRO, SER |
| 371 | 182 | ALA, GLY, SER |
| 372 | 183 | ALA, CYS, GLY, SER |
| 373 | 184 | ALA, GLY, SER |
| 374 | 185 | ALA, ARG, ASN, ASP, CYS, GLN, GLU, GLY, HIS, ILE, LEU, LYS, MET, PRO, SER, THR, TRP, VAL |
| 375 | 186 | ALA, ARG, ASN, ASP, CYS, GLN, GLU, GLY, HIS, LEU, LYS, MET, PHE, SER, THR, TRP, TYR, VAL |
| 376 | 187 | ALA, ASN, ASP, CYS, GLY, HIS, ILE, LEU, SER, THR, VAL |
| 377 | 188 | ALA, ARG, ASN, ASP, CYS, GLN, GLU, GLY, HIS, ILE, LEU, LYS, MET, SER, THR, VAL |
| 378 | 189 | ALA, GLY, SER |
| 379 | 190 | ALA, ASP, CYS, GLY, SER, THR |
| 380 | 191 | GLY |
| 381 | 192 | GLY |
| 382 | 193 | ALA, CYS, GLY, SER, THR |
| 383 | 194 | ALA, ARG, ASN, ASP, CYS, GLN, GLU, GLY, ILE, LEU, LYS, MET, SER, THR, VAL |
| 384 | 195 | ALA, ASN, ASP, CYS, GLN, GLU, GLY, LEU, SER, THR |
| 385 | 196 | ALA, ARG, ASN, ASP, CYS, GLN, GLU, GLY, HIS, ILE, LEU, LYS, MET, SER, THR, TRP, VAL |
| 386 | 197 | ALA, CYS, GLY, MET, SER, THR |
| 387 | 198 | ALA, ARG, ASN, ASP, CYS, GLN, GLU, GLY, HIS, ILE, LEU, LYS, MET, PHE, SER, THR, TRP, TYR, VAL, |
| 388 | 199 | ASN, ASP, GLY, LYS, SER |
| 389 | 200 | GLY |
| 390 | 201 | ALA, ARG, ASN, ASP, CYS, GLN, GLU, GLY, HIS, ILE, LEU, LYS, MET, PHE, SER, THR, TRP, TYR, VAL, |
| 391 | 202 | ALA, ASN, ASP, CYS, GLN, GLY, ILE, MET, PRO, SER, THR, VAL |
| 392 | 203 | ALA, ARG, ASN, ASP, CYS, GLN, GLU, GLY, HIS, LEU, LYS, MET, PHE, SER, THR, TRP, TYR |
| 393 | 204 | ALA, ARG, ASN, ASP, CYS, GLN, GLU, GLY, HIS, LEU, LYS, MET, PHE, SER, THR, TRP, TYR |
| 394 | 205 | ALA, CYS, GLN, GLU, GLY, SER, THR |
| 395 | 206 | ALA, ARG, ASN, ASP, CYS, GLN, GLU, GLY, ILE, LYS, MET, SER, THR, VAL |
| 396 | 207 | ALA, CYS, GLY, SER, THR, VAL |
| 397 | 208 | ALA, CYS, PHE, TRP, TYR |
| 398 | 209 | ARG, ASN, ASP, CYS, GLN, MET, SER |
| 399 | 210 | ALA, ARG, ASN, ASP, CYS, GLN, GLU, GLY, LEU, LYS, MET, SER |
| 400 | 211 | GLY |
| 401 | 212 | ALA, ARG, ASN, ASP, CYS, GLN, GLU, GLY, HIS, ILE, LEU, LYS, MET, PHE, PRO, SER, THR, TRP, TYR, |
| | | VAL |
| 402 | 213 | ALA, ARG, ASN, ASP, CYS, GLN, GLU, GLY, HIS, ILE, LEU, LYS, MET, PHE, SER, THR, TRP, TYR, VAL, |
| 403 | 214 | GLY |
| 404 | 215 | GLY |
| 405 | 216 | ALA, GLY, SER |
| 406 | 217 | ALA, CYS, GLY, SER, THR |
| 407 | 218 | GLY |
| 408 | 219 | GLY |
| 409 | 220 | GLY |
| 410 | 221 | ALA, ASN, CYS, GLY, ILE, SER, THR, VAL |
| 411 | 222 | ALA, GLY, SER |
| 412 | 223 | ALA, ARG, ASN, ASP, CYS, GLN, GLU, GLY, HIS, ILE, LEU, LYS, MET, SER, THR, VAL |
| 413 | 224 | ALA, ARG, ASN, ASP, CYS, GLN, GLU, GLY, ILE, LYS, MET, PHE, SER, THR, TYR, VAL |
| 414 | 225 | ALA, ASN, CYS, GLN, GLU, HIS, ILE, LEU, LYS, MET, PHE, SER, THR, TYR, VAL |
| 415 | 226 | ALA, ARG, ASN, ASP, CYS, GLN, GLU, GLY, HIS, ILE, LEU, LYS, MET, PHE, PRO, SER, THR, TRP, TYR, |
| | | VAL |
| 416 | 227 | ALA, ARG, ASN, ASP, CYS, GLN, GLU, GLY, ILE, LEU, LYS, MET, PRO, SER, THR, VAL |
| 417 | 228 | ALA, CYS, GLN, GLU, GLY, MET, PRO, SER, THR |
| 418 | 229 | ALA, ARG, ASN, ASP, CYS, GLN, GLU, GLY, HIS, ILE, LEU, LYS, MET, PHE, PRO, SER, THR, TRP, TYR, |
| | | VAL |
| 419 | 230 | ALA, ASN, ASP, CYS, GLN, GLU, GLY, HIS, LEU, PHE, SER, TRP, TYR |
| 420 | 231 | GLN, GLU |
| 421 | 232 | ALA, ARG, ASN, ASP, CYS, GLN, GLU, GLY, HIS, ILE, LEU, LYS, MET, PHE, SER, THR, TRP, TYR, VAL, |
| 422 | 233 | ALA, ARG, ASN, ASP, CYS, GLN, GLU, GLY, HIS, ILE, LEU, LYS, MET, PHE, PRO, SER, THR, TRP, TYR, |
| | | VAL |
| 423 | 234 | ALA, GLY, SER |
| 424 | 235 | ALA, ARG, ASN, ASP, CYS, GLN, GLU, GLY, HIS, ILE, LEU, LYS, MET, PHE, SER, THR, TRP, TYR, VAL, |
| 425 | 236 | ALA, CYS, GLY, PRO, SER, THR, VAL |
| 426 | 237 | ALA, ARG, ASN, ASP, CYS, GLN, GLU, GLY, HIS, ILE, LEU, LYS, MET, PHE, PRO, SER, THR, TRP, TYR, |
| | | VAL |
| 427 | 238 | ALA, ARG, ASN, ASP, CYS, GLN, GLU, GLY, HIS, LYS, MET, PHE, PRO, SER, THR, TRP, TYR, VAL |
| 428 | 239 | ALA, ASN, ASP, CYS, GLN, GLU, GLY, SER, THR, VAL |
| 429 | 240 | ALA, ASN, ASP, CYS, GLY, SER |
| 430 | 241 | ALA, ASN, ASP, CYS, GLY, SER, THR |
| 431 | 242 | ALA, ARG, ASN, ASP, CYS, GLN, GLU, GLY, HIS, ILE, LEU, LYS, MET, PHE, PRO, SER, THR, TRP, TYR, |
| | | VAL |
| 432 | 243 | ALA, ARG, ASN, ASP, CYS, GLN, GLU, GLY, HIS, ILE, LEU, LYS, MET, PHE, PRO, SER, THR, TRP, TYR, |
| | | VAL |
| 433 | 244 | ALA, ARG, ASN, ASP, CYS, GLN, GLU, GLY, HIS, ILE, LEU, LYS, MET, PHE, SER, THR, TRP, TYR, VAL, |
| 434 | 245 | ALA, ARG, ASN, ASP, CYS, GLN, GLU, GLY, HIS, ILE, LEU, LYS, MET, PHE, PRO, SER, THR, TRP, TYR, |
| | | VAL |
| 435 | 246 | ALA, ARG, ASN, ASP, CYS, GLN, GLU, GLY, HIS, ILE, LEU, LYS, MET, PHE, PRO, SER, THR, TRP, TYR, |
| | | VAL |
| 436 | 247 | GLY |
| 437 | 248 | ALA, ARG, ASN, CYS, GLN, SER, THR |
| 438 | 249 | GLY |
| 439 | 250 | ALA, ASN, ASP, CYS, GLN, GLU, GLY, ILE, MET, SER, THR, VAL |
| 440 | 251 | ALA, GLY, PRO, SER |
| 441 | 252 | ASP |
| 442 | 253 | ALA, ASN, ASP, CYS, GLN, GLU, GLY, LEU, MET, SER, THR |
| 443 | 254 | ALA, GLY, SER |
| 444 | 255 | ALA, GLY |
| 445 | 256 | ALA, ASN, ASP, CYS, GLY, SER |
| 446 | 257 | ALA, GLY, SER |
| 447 | 258 | ALA, ASN, ASP, CYS, GLY, SER, THR |
| 448 | 259 | ALA, ARG, ASN, ASP, CYS, GLN, GLU, GLY, HIS, ILE, LEU, LYS, MET, PHE, PRO, SER, THR, TRP, TYR, |
| | | VAL |
| 449 | 260 | ALA, ARG, ASN, ASP, CYS, GLN, GLU, GLY, HIS, ILE, LEU, LYS, MET, PHE, SER, THR, TRP, TYR, VAL, |
| 450 | 261 | ALA, ASN, ASP, CYS, GLY, HIS, SER, THR |
| 451 | 262 | GLY |
| 452 | 263 | ALA, ASN, CYS, GLN, HIS, ILE, LEU, PHE, SER, THR, TYR, VAL |
| 453 | 264 | ALA, ARG, ASN, ASP, CYS, GLN, GLU, GLY, HIS, ILE, LEU, LYS, MET, PHE, SER, THR, TRP, TYR, VAL, |
| 454 | 265 | ALA, ASN, ASP, CYS, GLY, SER, THR, VAL |
| 455 | 266 | ALA, ARG, ASN, ASP, CYS, GLU, GLY, HIS, ILE, MET, PHE, SER, THR, TRP, TYR, VAL |
| 456 | 267 | ALA, ASN, ASP, CYS, GLN, GLU, GLY, HIS, ILE, LEU, SER, THR, VAL |
| 457 | 268 | ALA, ASN, ASP, CYS, GLY, ILE, MET, SER, THR, TRP, VAL |
| 458 | 269 | ALA, ARG, ASN, ASP, CYS, GLN, GLU, GLY, LYS, MET, SER |
| 459 | 270 | ALA, ARG, ASN, ASP, CYS, GLN, GLU, GLY, HIS, ILE, LEU, LYS, MET, PHE, SER, THR, TRP, TYR, VAL, |
| 460 | 271 | ALA, ARG, ASN, ASP, CYS, GLN, GLU, GLY, HIS, ILE, LEU, LYS, MET, PHE, PRO, SER, THR, TYR, VAL, |
| 461 | 272 | ALA, ASN, ASP, CYS, GLN, GLY, HIS, LYS, MET, SER, THR |
| 462 | 273 | ALA, ARG, ASN, ASP, CYS, GLN, GLY, HIS, ILE, LYS, MET, PHE, SER, THR, TRP, TYR, VAL |
| 463 | 274 | ALA, ARG, ASN, ASP, CYS, GLN, GLU, GLY, HIS, ILE, LEU, LYS, MET, PHE, SER, THR, TRP, TYR, VAL, |
| 464 | 275 | GLY |
| 465 | 276 | GLY |
| 466 | 277 | ALA, ASN, ASP, CYS, GLY, SER, THR |
| 467 | 278 | SER |
| 468 | 279 | ALA, ASP, CYS, GLY, SER |
| 469 | 280 | ALA, ASN, ASP, CYS, GLY, SER, THR, VAL |
| 470 | 281 | ALA, GLY, SER |
| 471 | 282 | ALA, CYS, GLY, PRO, SER |
| 472 | 283 | ALA, ASN, ASP, CYS, GLN, GLU, GLY, HIS, LEU, MET, SER, THR, VAL |
| 473 | 284 | ALA, ASN, ASP, CYS, GLN, GLU, GLY, HIS, ILE, LEU, LYS, MET, PHE, SER, THR, TRP, TYR, VAL |
| 474 | 285 | ALA, GLY, SER |
| 475 | 286 | ALA, GLY, SER |
| 476 | 287 | ALA, ARG, ASN, ASP, CYS, GLN, GLU, GLY, HIS, LEU, LYS, MET, SER, THR, VAL |
| 477 | 288 | ALA, ASN, ASP, CYS, GLN, GLU, GLY, HIS, ILE, LYS, MET, SER, THR, VAL |
| 478 | 289 | ALA, GLY, SER |
| 479 | 290 | ALA, ARG, ASN, ASP, CYS, GLN, GLU, GLY, HIS, ILE, LEU, LYS, MET, SER, THR, TRP, TYR |
| 480 | 291 | ALA, ARG, ASN, ASP, CYS, GLU, GLY, ILE, LEU, LYS, MET, SER, THR, VAL |
| 481 | 292 | ALA, ASN, ASP, CYS, GLN, GLU, GLY, MET, SER |
| 482 | 293 | ALA, GLN, GLU, HIS, LYS, THR |
| 483 | 294 | ALA, ARG, ASN, ASP, CYS, GLN, GLU, GLY, HIS, LYS, MET, PHE, SER, TRP, TYR |
| 484 | 295 | ALA, ARG, ASN, ASP, CYS, GLN, GLU, GLY, ILE, LEU, LYS, MET, SER, THR, VAL |
| 485 | 296 | ALA, ARG, ASN, ASP, CYS, GLN, GLU, GLY, HIS, ILE, LEU, LYS, MET, PHE, SER, TRP, TYR, VAL |
| 486 | 297 | ALA, ARG, ASN, ASP, CYS, GLN, GLU, GLY, HIS, ILE, LEU, LYS, MET, PHE, SER, THR, TRP, TYR, VAL |
| 487 | 298 | ALA, ARG, ASN, ASP, CYS, GLN, GLU, GLY, HIS, LEU, LYS, MET, PRO, SER, THR, TRP, VAL |
| 488 | 299 | ALA, ARG, ASN, ASP, CYS, GLN, GLU, GLY, ILE, LEU, LYS, MET, SER, THR, VAL |
| 489 | 300 | GLY |
| 490 | 301 | ALA, ARG, ASN, ASP, CYS, GLN, GLU, GLY, HIS, LEU, LYS, MET, PHE, PRO, SER, THR, TRP, TYR, VAL |
| 491 | 302 | ALA, ARG, ASN, ASP, CYS, GLN, GLU, GLY, HIS, ILE, LEU, LYS, MET, PHE, PRO, SER, THR, VAL |
| 492 | 303 | ALA, ARG, ASN, ASP, CYS, GLN, GLU, GLY, HIS, ILE, LEU, LYS, MET, PRO, SER, THR, VAL |
| 493 | 304 | ALA, ARG, ASN, ASP, CYS, GLN, GLU, GLY, HIS, ILE, LYS, MET, PHE, PRO, SER, THR, TRP, TYR, VAL |
| 494 | 305 | ALA, ARG, ASN, ASP, CYS, GLN, GLU, GLY, HIS, ILE, LEU, LYS, MET, PHE, PRO, SER, THR, TRP, TYR, |
| | | VAL |
| 495 | 306 | ALA, ASN, ASP, CYS, GLN, GLU, GLY, HIS, LEU, MET, SER, THR |
| 496 | 307 | ALA, HIS, PHE, SER, THR, TYR |
| 497 | 308 | ALA, ARG, ASN, ASP, CYS, GLN, GLU, GLY, HIS, ILE, LEU, LYS, MET, PHE, SER, THR, TRP, TYR |
| 498 | 309 | ALA, ARG, ASN, ASP, CYS, GLN, GLU, GLY, LEU, MET, SER, THR |
| 499 | 310 | ALA, ARG, ASN, ASP, CYS, GLN, GLU, GLY, HIS, LEU, LYS, MET, PHE, PRO, SER, THR, TRP, TYR |
| 500 | 311 | ALA, ARG, ASN, ASP, CYS, GLN, GLU, GLY, HIS, ILE, LEU, LYS, MET, PHE, PRO, SER, THR, TRP, TYR, |
| | | VAL |
| 501 | 312 | ALA, ARG, ASN, ASP, CYS, GLN, GLU, GLY, HIS, ILE, LEU, LYS, MET, PHE, SER, THR, TRP, TYR, VAL |
| 502 | 313 | ALA, ASN, ASP, CYS, GLY, ILE, MET, SER, THR, VAL |
| 503 | 314 | ALA, ASN, ASP, CYS, HIS, LEU, MET, PHE, SER, THR, TYR, VAL |
| 504 | 315 | ALA, ARG, ASN, ASP, CYS, GLN, GLU, GLY, HIS, ILE, LYS, MET, PHE, SER, THR, TRP, TYR, VAL |
| 505 | 316 | ALA, ARG, ASN, ASP, CYS, GLN, GLU, GLY, HIS, ILE, LEU, LYS, MET, PRO, SER, THR, TRP, VAL |
| 506 | 317 | ALA, ASN, ASP, CYS, GLN, GLY, ILE, SER, THR, VAL |
| 507 | 318 | ALA, ARG, ASN, ASP, CYS, GLN, GLU, GLY, HIS, ILE, LYS, MET, PHE, SER, THR, TRP, TYR, VAL |
| 508 | 319 | ALA, ARG, ASN, ASP, CYS, GLN, GLU, GLY, HIS, ILE, LEU, LYS, MET, PHE, SER, THR, TRP, TYR, VAL |
| 509 | 320 | GLY |
| 510 | 321 | ALA, ARG, ASN, ASP, CYS, GLN, GLU, GLY, HIS, LYS, MET, SER, THR, TRP, TYR |
| 511 | 322 | ALA, ASN, ASP, CYS, GLY, SER |
| 512 | 323 | ALA, ASN, ASP, CYS |
| 513 | 324 | ALA, ASN, ASP, CYS, GLN, GLU, GLY, ILE, LYS, MET, SER, THR, VAL |
| 514 | 325 | ALA, ARG, ASN, ASP, CYS, GLN, GLU, GLY, HIS, ILE, LYS, MET, PHE, SER, THR, TRP, TYR, VAL |
| 515 | 326 | ALA, ARG, ASN, ASP, CYS, GLN, GLU, GLY, HIS, LYS, MET, SER |
| 516 | 327 | ALA, ARG, ASN, ASP, CYS, GLN, GLU, GLY, HIS, ILE, LEU, LYS, MET, PHE, PRO, SER, THR, TRP, TYR, |
| | | VAL |
| 517 | 328 | ALA, ARG, ASN, ASP, CYS, GLN, GLU, GLY, HIS, ILE, LEU, LYS, MET, PHE, SER, THR, TRP, TYR, VAL |
| 518 | 329 | ALA, ARG, ASN, ASP, CYS, GLN, GLU, GLY, HIS, LEU, LYS, MET, PHE, SER, THR, TRP, TYR |
| 519 | 330 | ALA, ARG, ASN, ASP, CYS, GLN, GLU, GLY, HIS, ILE, LEU, LYS, MET, PHE, SER, THR, TRP, TYR, VAL |
| 520 | 331 | HIS, PHE, THR, TRP, TYR |
| 521 | 332 | ALA, ARG, ASN, ASP, CYS, GLN, GLU, GLY, HIS, ILE, LEU, LYS, MET, PHE, SER, THR, TYR, VAL |
| 522 | 333 | ALA, GLY, SER |
| 523 | 334 | CYS, GLY, HIS, LYS, MET, PHE, SER, TYR |
| 524 | 335 | ALA, ARG, ASN, ASP, CYS, GLN, GLU, GLY, HIS, ILE, LEU, LYS, MET, PHE, PRO, SER, THR, TRP, TYR, |
| | | VAL |
| 525 | 336 | GLY |
| 526 | 337 | HIS, PHE, TRP |
| 527 | 338 | ALA, ASN, ASP, CYS, SER |
| 528 | 339 | ALA, GLY, PRO, SER |
| 529 | 340 | ALA, ASP, CYS, GLY, SER, THR |
| 530 | 341 | ALA, ASN, CYS, GLY, SER, THR, VAL |
| 531 | 342 | GLY |
| 532 | 343 | ALA, ARG, ASN, ASP, CYS, GLN, GLU, GLY, LEU, LYS, MET, SER |
| 533 | 344 | GLY |
| 534 | 345 | ALA, CYS, GLY, SER, THR |
| 535 | 346 | ALA, CYS, GLY, PRO, SER, THR |
| 536 | 347 | ALA, ARG, ASN, ASP, CYS, GLN, GLU, GLY, ILE, LEU, LYS, MET, PHE, SER, THR, TYR, VAL |
| 537 | 348 | GLY |
| 538 | 349 | ALA, ARG, ASN, ASP, CYS, GLN, GLU, GLY, HIS, ILE, LEU, LYS, MET, PHE, SER, THR, TRP, TYR, VAL |
| 539 | 350 | ALA, ARG, ASN, ASP, CYS, GLN, GLU, GLY, HIS, LEU, LYS, MET, PHE, SER, THR, TRP, TYR |
| 540 | 351 | ALA, ASN, ASP, CYS, GLN, GLU, GLY, LEU, LYS, SER, THR, VAL |
| 541 | 352 | ALA, ARG, ASN, ASP, CYS, GLN, GLU, GLY, LEU, LYS, MET, SER, THR |
| 542 | 353 | ALA, ARG, ASN, ASP, CYS, GLN, GLU, GLY, HIS, ILE, LEU, LYS, MET, PHE, SER, THR, TRP, TYR, VAL |
| 543 | 354 | ALA, ARG, CYS, GLN, GLU, GLY, MET, SER, THR |
| 544 | 355 | ALA, ASN, ASP, CYS, GLN, GLU, GLY, LEU, MET, SER, THR |
| 545 | 356 | ALA, ARG, ASN, ASP, CYS, GLN, GLU, GLY, HIS, ILE, LEU, LYS, MET, PHE, SER, THR, TRP |
| 546 | 357 | Any residue |
| 547 | 358 | Any residue |
| 548 | 359 | Any residue |
| 549 | 360 | Any residue |
| 550 | 361 | Any residue |
| 551 | 362 | Any residue |
| 552 | 363 | Any residue |
| 553 | 364 | Any residue |

In some aspects, a polypeptide sequences disclosed herein further comprises a histidine tag. In some aspects, the histidine tag is fused to the polypeptide at the C-terminus of the polypeptide. Any suitable histidine tag can be used. In some aspects, the histidine tag is linked to a TEV protease cut site (ENLYFQS) (SEQ ID NO: 18) to allow for its efficient removal with TEV protease after purification, for example, the tag may comprise or consist of the amino acid sequence GSTENLYFQSGALEHHHHHH (SEQ ID NO: 17). In another aspect, a cleavable histidine tag is incorporated at the C-terminus of the polypeptide sequence, comprising the amino acid sequence X_{N}PQ(L/Q)PX_{N}HHHHHH (SEQ ID NO: 15), wherein X_{N} is an linker of between 1-25 amino acid residues. In one non-limiting example, the cleavable histidine tag comprises the amino acid sequence

(SEQ ID NO: 16)
GSSGSSGSQPQLPYGSSGSSGSHHHHHH.

As illustrated in Table 5, point substitutions relative to the Kuma010/011 amino acid sequence can affect catalytic activity. Table 5 lists the effectiveness of individual mutations in catalyzing the degradation of various gliadin peptide sequences. The examples disclosed in WO2016/200880 provide further data regarding specific individual and combination mutants.

TABLE 5

| | | | | % | % |
|---|---|---|---|---|---|
| Position | | | A.A. | Improvement on | Improvement on |
| (Full | Position | Kuma010 | relative to | PFPQPQLPY | PFPQPQQPF |
| Length) | (Truncated) | A.A. | Kuma010/011 | (SEQ ID NO: 11) | (SEQ ID NO: 12) |
| 221 | 32 | E | D, N, Q, H | 105% | ND |
| 262 | 73 | K | E | 109% | 110% |
| 268 | 79 | V | A | 107% | 89% |
| 268 | 79 | V | S | 104% | 83% |
| 268 | 79 | V | T | 127% | 105% |
| 269 | 80 | E | L | 113% | 84% |
| 269 | 80 | E | T | 263% | 191% |
| 270 | 81 | L | A | 203% | 92% |
| 270 | 81 | L | T | 307% | 29% |
| 270 | 81 | L | V | 474% | 61% |
| 319 | 130 | S | A | 154% | 184% |
| 354 | 165 | S | A | 152% | 140% |
| 354 | 165 | S | E | 124% | 120% |
| 354 | 165 | S | Q | 145% | 141% |
| 354 | 165 | S | R | 109% | 82% |
| 354 | 165 | S | Y | 46% | 105% |
| 358 | 169 | G | N | 120% | 99% |
| 358 | 169 | G | S | 331% | 224% |
| 358 | 169 | G | Q | 147% | 149% |
| 358 | 169 | G | T | 283% | 128% |
| 368 | 179 | H | F | 334% | 104% |
| 368 | 179 | H | Q | 199% | 195% |
| 399 | 210 | D | Q | 149% | 208% |
| 402 | 213 | D | S | 94% | 108% |
| 402 | 213 | D | Q | 164% | 111% |
| 406 | 217 | T | S | 84% | 101% |
| 424 | 235 | N | K | 285% | ND |
| 449 | 260 | A | E | 149% | 208% |
| 449 | 260 | A | N | 119% | 118% |
| 461 | 272 | T | R | 120% | 86% |
| 463 | 274 | I | A | 51% | 234% |
| 463 | 274 | I | L | 124% | 22% |
| 463 | 274 | I | M | 123% | 53% |
| 463 | 274 | I | Q | 129% | 69% |
| 463 | 274 | I | R | 29% | 110% |
| 463 | 274 | I | T | 130% | 239% |
| 463 | 274 | I | V | 256% | 141% |

In certain aspects, the present disclosure provides polypeptides that include at least one mutation that improves production of the polypeptide. In some aspects, mutations that improve production provide improvements in one of three categories: 1. altering purification method; 2. increase in yield; and 3. decreasing the probability that enzymatic self-processing would occur during purification, thereby simplifying analysis. Addition of a His tag that is removable by the proteolytic activity of the polypeptides disclosed herein falls into category 1; the R105H mutant appears to improve yield by ˜2-fold, placing this mutation into category 2; and mutations in positions 171-174 place these mutants into category 3.

As used throughout the present application, the term “polypeptide” is used in its broadest sense to refer to a sequence of subunit amino acids, whether naturally occurring or of synthetic origin. The polypeptides of the disclosure may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, or glycosylation. Such linkage can be covalent or non-covalent as is understood by those of skill in the art. In some aspects, the polypeptides are linked to any other suitable linkers, including but not limited to any linkers that can be used for purification or detection (such as FLAG or His tags).

### A. Nucleic Acids

In another aspect, the present disclosure provides isolated nucleic acids encoding the polypeptide of any aspect of the disclosure. An exemplary nucleic acid that encodes the Kuma062-M is shown below:

SEQ ID NO: 22
AGTGATATGGAAAAACCGTGGAAAGAAGGTGAAGAAGCCCGCGCAGTGC
TGCAAGGTCATGCTCGTGCGCAGGCACCGCAAGCAGTCGATAAAGGCCC
GGTGGCAGGTGACGAACGCATGGCTGTTACCGTGGTTCTGCGTCGCCAG
CGTGCAGGTGAACTGGCGGCCCACGTGGAACGTCAAGCAGCTATTGCTC
CGCATGCGCGCGAACACCTGAAACGTGAAGCGTTTGCGGCCAGTCATGG
TGCGTCCCTGGATGACTTTGCCGAACTGCGTCGCTTCGCAGATGCTCAC
GGCCTGGCGCTGGACCGTGCAAACGTTGCAGCTGGCACCGCCGTTCTGT
CTGGTCCGGACGATGCAATCAATCGCGCTTTTGGTGTGGAACTGCGTCA
TTTCGATCACCCGGACGGCTCATATCGTTCGTACCTGGGTGAAGTCACC
GTGCCGGCCAGTATTGCACCGATGATCGAAGCGGTTCTGGGCCTGGATA
CGCGTCCGGTCGCCCGCCGTCGTTTTCGTATGCAGCGTCGCGCAGAAGG
CGGTTTCGAAGCTCGTTCCCAAGCGGCGGCACCGACCGCATATACGCCG
CTGGATGTTGCGCAGGCCTACCAATTTCCGGAAGGTCTGGACGGCCAGG
GTCAATGCATTGCCATTATCGAACTGGGCGGTGGCTATGATGAAGCTTC
ACTGGCGCAGTACTTCGCGTCGCTGGGCGTGCCGGCACCGCAAGTCGTG
AGTGTTTCCGTCGATGGTGCGAGCAACCAGCCGACCGGTGATCCGGAAG
GTCCGGACGGTGAAGTGACCCTGGATATCGAAGTTGCAGGCGCTCTGGC
GCCGGGTGCCAAATTTGCAGTGTATTTCGCGCCGGATACCACTGCCGGT
TTTCTGGACGCGATTACCACGGCCATCCACGATCCGACGCTGAAACCGA
GCGTTGTCTCAATTTCGTGGAGCATGCCGGAAGACAGCTGGACCTCTGC
TGCGATCGCCGCAATGAACCGTGCGTTTCTGGATGCTGCGGCCCTGGGT
GTGACCGTTCTGGCAGCTGCGGGCGACCAGGGTTCTACGAGCGGCGAAC
AGGACGGTCTGTATCATGTGCATTTCCCGGCCGCATCACCGTACGTTCT
GGCGTGCGGTGGCACGCGCCTGGTCGCATCGGGTGGCCGTATTGCGCAG
GAAACCGTCTGGAACCAGGGTCCGGACGGTGGTGCAACGGGTGGCGGTG
TGAGCCGCATCTTCCCGCTGCCGGCATGGCAGGAACACGCTAACGTTCC
GCCGTCTGCAAATCCGGGCGCGAGCAGCGGCCGTGGTGTCCCGGATCTG
GCTGGTAATGCGGACCCGCAGACCGGTTATGAAGTGGTTATTGATGGCG
AAGCAACCGTCACCGGCGGTACGAGCGCCGTGGCACCGCTGTTTGCTGC
GCTGGTTGCGCGTATTAACCAGAAACTGGGCAAAGCAGTTGGTTATCTG
AATCCGACCCTGTACCAACTGCCGGCAGATGTTTTCCATGACATCACGG
AGGGTAACAATGATATTGCAAACCGTGCGCAGATTTATCAAGCAGGTCC
GGGCTGGGACCCGTGTACCGGTCTGGGTTCACCGATTGGTGTGCGTCTG
CTGCAAGCACTGTTGCCGAGTGCCTCCCAGCCGCAACCGTGA

The isolated nucleic acid sequence may comprise RNA or DNA. As used herein. “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to poly A sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the present disclosure provides nucleic acid expression vectors comprising the isolated nucleic acid of any aspect of the disclosure operatively linked to a suitable control sequence. “Recombinant expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example. intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E.J. Murray, The Humana Press Inc., Clifton, N.J.), and the Ambion 1998 Catalog (Ambion, Austin, TX). The expression vector must be replicable in the host organisms either as an episome or by integration into host chromosomal DNA. In a preferred aspect, the expression vector comprises a plasmid. However, the disclosure is intended to include other expression vectors that serve equivalent functions, such as viral vectors.

### B. Host Cells

In another aspect, the present disclosure provides recombinant host cells comprising the nucleic acid expression vectors of the disclosure. Any host cell capable of producing a recombinant protein can be used in the methods disclosed herein. The host cells can be either prokaryotic or eukaryotic. In some aspects, the host cell is a prokaryotic cell. Non-limiting examples of suitable prokaryotic host cells include Escherichia coli, Bacillus subtilis, Caulobacter crescentus, Rodhobacter sphaeroides, Pseudoalteromonas haloplanktis, Shewanella sp, strain Ac10, Pseudomonas fluorescensi Pseudomonas putida, Pseudomonas aeruginosa, Halomonas elongata, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, and Lactobacillus gasseri. In some aspects, the host cell is a eukaryotic cell. Non-limiting examples of suitable eukaryotic host cells include Saccharomyces cerevisiae and Aspergillus nidulans. The cells can be transiently or stably transfected or transduced. Such transfection and transduction of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique. 2^{nd} Ed. (R.I. Freshney. 1987. Liss, Inc. New York, NY). A method of producing a polypeptide according to the disclosure is an additional part of the disclosure. The method comprises the steps of (a) culturing a host according to this aspect of the disclosure under conditions conducive to the expression of the polypeptide, and (b) optionally, recovering the expressed polypeptide. The expressed polypeptide can be recovered from the cell free extract, cell pellet, or recovered from the culture medium. Methods to purify recombinantly expressed polypeptides are well known to the man skilled in the art.

### C. Pharmaceutical Compositions

In a further aspect, the present disclosure provides pharmaceutical compositions, comprising the polypeptide, nucleic acid, nucleic acid expression vector, and/or the recombinant host cell of any aspect or aspect of the disclosure, and a pharmaceutically acceptable carrier. The pharmaceutical compositions of the disclosure can be used, for example, in the methods of the disclosure described below. The pharmaceutical composition may comprise in addition to the polypeptides, nucleic acids, etc. of the disclosure (a) a lyoprotectant; (b) a surfactant; (c) a bulking agent; (d) a tonicity adjusting agent; (e) a stabilizer; (f) a preservative and/or (g) a buffer.

In some aspects, the buffer in the pharmaceutical composition is a Tris buffer, a histidine buffer, a phosphate buffer, a citrate buffer or an acetate buffer. The pharmaceutical composition may also include a lyoprotectant, e.g., sucrose, sorbitol or trehalose. In certain aspects, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other aspects, the pharmaceutical composition includes a bulking agent, like glycine. In yet other aspects, the pharmaceutical composition includes a surfactant e.g., polysorbate-20, polysorbate-40, polysorbate-60, polysorbate-65, polysorbate-80) polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other aspects, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptides, nucleic acids, etc. of the disclosure may be the sole active agent in the pharmaceutical composition, or the composition may further comprise one or more other active agents suitable for an intended use.

The pharmaceutical compositions described herein generally comprise a combination of a compound described herein and a pharmaceutically acceptable carrier, diluent, or excipient. Such compositions are substantially free of non-pharmaceutically acceptable components, i.e., contain amounts of non-pharmaceutically acceptable components lower than permitted by US regulatory requirements at the time of filing this application. In some aspects of this aspect, if the compound is dissolved or suspended in water, the composition further optionally comprises an additional pharmaceutically acceptable carrier, diluent, or excipient. In other aspects, the pharmaceutical compositions described herein are solid pharmaceutical compositions (e.g., tablet, capsules, etc.).

The compositions described herein could also be provided as a dietary supplement as described by the US regulatory agencies.

These compositions can be prepared in a manner well known in the pharmaceutical art, and can be administered by any suitable route. In a preferred aspect, the pharmaceutical compositions and formulations are designed for oral administration. Conventional pharmaceutical carriers, aqueous, powder or oily bases, thickeners and the like may be necessary or desirable.

The pharmaceutical compositions can be in any suitable form, including but not limited to tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

### 3. Methods of the Disclosure

In another aspect, the present disclosure provides methods for treating celiac sprue or non-celiac gluten sensitivity (NCGS), comprising administering to an individual with celiac sprue or NCGS an amount effective to treat the celiac sprue or NCGS of one or more polypeptides selected from the group consisting of the polypeptides of the of the disclosure, or using one or more of these polypeptides to process food for consumption by individuals with celiac sprue or NCGS.

In certain aspects, the method comprises administering to a subject affected with celiac sprue or NCGS a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1. In certain aspects. the method comprises administering to a subject affected with celiac sprue or NCGS a polypeptide comprising an amino acid sequence having at least about 90% sequence identity to the amino acid sequence set forth in SEQ ID NO: 1. In certain aspects, the method comprises administering to a subject affected with celiac sprue or NCGS a polypeptide comprising an amino acid sequence having at least about 95% sequence identity to the amino acid sequence set forth in SEQ ID NO: 1. In certain aspects, the method comprises administering to a subject affected with celiac sprue or NCGS a polypeptide comprising an amino acid sequence having at least about 96% sequence identity to the amino acid sequence set forth in SEQ ID NO: 1. In certain aspects, the method comprises administering to a subject affected with celiac sprue or NCGS a polypeptide comprising an amino acid sequence having at least about 97% sequence identity to the amino acid sequence set forth in SEQ ID NO: 1. In certain aspects, the method comprises administering to a subject affected with celiac sprue or NCGS a polypeptide comprising an amino acid sequence having at least about 98% sequence identity to the amino acid sequence set forth in SEQ ID NO: 1. In certain aspects, the method comprises administering to a subject affected with celiac sprue or NCGS a polypeptide comprising an amino acid sequence having at least about 99% sequence identity to the amino acid sequence set forth in SEQ ID NO: 1. In certain aspects, the method comprises administering to a subject affected with celiac sprue or NCGS a polypeptide comprising the amino acid sequence set forth in SEQ ID NO: 1.

In certain aspects, the method comprises administering to a subject affected with celiac sprue or NCGS a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 8. In certain aspects. the method comprises administering to a subject affected with celiac sprue or NCGS a polypeptide comprising an amino acid sequence having at least about 90% sequence identity to the amino acid sequence set forth in SEQ ID NO: 8. In certain aspects, the method comprises administering to a subject affected with celiac sprue or NCGS a polypeptide comprising an amino acid sequence having at least about 95% sequence identity to the amino acid sequence set forth in SEQ ID NO: 8. In certain aspects, the method comprises administering to a subject affected with celiac sprue or NCGS a polypeptide comprising an amino acid sequence having at least about 96% sequence identity to the amino acid sequence set forth in SEQ ID NO: 8. In certain aspects, the method comprises administering to a subject affected with celiac sprue or NCGS a polypeptide comprising an amino acid sequence having at least about 97% sequence identity to the amino acid sequence set forth in SEQ ID NO: 8. In certain aspects, the method comprises administering to a subject affected with celiac sprue or NCGS a polypeptide comprising an amino acid sequence having at least about 98% sequence identity to the amino acid sequence set forth in SEQ ID NO: 8. In certain aspects, the method comprises administering to a subject affected with celiac sprue or NCGS a polypeptide comprising an amino acid sequence having at least about 99% sequence identity to the amino acid sequence set forth in SEQ ID NO: 8. In certain aspects, the method comprises administering to a subject affected with celiac sprue or NCGS a polypeptide comprising the amino acid sequence set forth in SEQ ID NO: 8.

In certain aspects, the method comprises administering to a subject affected with celiac sprue or NCGS a polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1; wherein the polypeptide comprises the amino acid sequence set forth in SEQ ID NO: 8; and wherein the polypeptide comprises a Ser at the amino acid residue corresponding to amino acid 278 in SEQ ID NO: 3, a Glu at the amino acid residue corresponding to amino acid 78 in SEQ ID NO: 3, and an Asp at the amino acid residue corresponding to amino acid 82 in SEQ ID NO: 3.

In certain aspects, the disclosure provides a method for degrading gluten in a food item, comprising contacting the food item with an amount effective to degrade the gluten with the polypeptide described above herein, thereby degrading the gluten in the food item. In certain aspects, the disclosure provides a method for degrading gluten in a food item, comprising contacting the food item with an amount effective to degrade the gluten with the the pharmaceutical composition described above herein, thereby degrading the gluten in the food item.

In certain aspects, the disclosure provides a method for degrading gliadin in a food item, comprising contacting the food item with an amount effective to degrade the gliadin with the polypeptide or the pharmaceutical composition described herein, thereby degrading the gluten in the food item. In some aspects, the method degrades at least about 75%, at least about 80%, at least about 85%, at least about 90%, at least about 95%, at least about 96%, at least about 98%, at least about 99%, or about 100% of the gluten or gliadin in the food item. In some aspects, the methods disclosed herein can degrade gluten or gliadin in a food item in less than about 1.5 hours, less than about 1 hour, less than about 45 minutes, less than about 40 minutes, less than about 30 minutes, less than about 25 minutes, less than about 20 minutes, less than about 15 minutes, less than about 10 minutes, or less than about 5 minutes. In some aspects, the methods disclosed here can degrade gluten or gliadin in a food item under a pH value less than about 6.5, less than about 6.0, less than about 5.5, less than about 5.0, less than about 4.5, less than about 4.0, less than about 3.5, or less than about 3.0.

The inventors of the present disclosure have discovered that the polypeptides of the disclosure are capable of degrading proline (P)- and glutamine (Q)-rich components of gluten known as ‘gliadins’ believed responsible for the bulk of the immune response in most celiac sprue patients. The polypeptides of the present disclosure show superior activity in degrading peptides having a PQLP (SEQ ID NO: 9) or PQQP (SEQ ID NO: 10) motif (such as PFPQPQLPY (SEQ ID NO: 11) and/or PFPQPQQPF (SEQ ID NO: 12)), which are substrates representative of gliadin) at pH 4 compared to Kuma010/011 and other polypeptides disclosed as useful for treating celiac sprue (WO2015/023728). Thus, the polypeptides of the disclosure constitute significantly improved therapeutics for treating celiac sprue and NCGS.

In a certain aspect, the pharmaceutical composition and/or formulation of a polypeptide disclosed herein is administered orally. Non-limiting examples of routes of oral administration include the use of tablets, pills, lozenges, elixirs, suspensions, emulsions, solutions, syrups, or any combination thereof. In certain aspects, a pharmaceutical composition comprising a polypeptide disclosed herein is administered to a subject before the subject ingests a substance, e.g., food, comprising one or more gluten protein. In some aspects, a pharmaceutical composition comprising a polypeptide disclosed herein is administered to a subject at the same time the subject ingests a substance, e.g., food, comprising one or more gluten protein. In some aspects, a pharmaceutical composition comprising a polypeptide disclosed herein is administered to a subject after the subject ingests a substance, e.g., food, comprising one or more gluten protein.

Dosage regimens can be adjusted to provide the optimum desired response (e.g., a therapeutic or prophylactic response). A suitable dosage range may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. The polypeptides can be delivered in a single bolus, or may be administered more than once (e.g., 2, 3, 4, 5, or more times) as determined by an attending physician.

The present disclosure is further illustrated by the following examples, which should not be construed as limiting. All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1: Degradation of Gluten in Whole Bread by Kuma062-M

This study is to demonstrate that Kuma062-M can effectively degrade gluten.

Laboratory simulations of gastric digestions were designed to represent gastric digestion in humans. Bread samples were first mashed in artificial saliva to simulate mastication, then acidified by the addition of hydrochloric acid. Unless otherwise indicated, the pH of the gastric digestion was 3.6-4.5. Samples were blended to ensure ability to draw up an appropriate representation of material through a narrow pipette tip (since the ELISA methods utilize very small volumes by necessity); however, where indicated, samples were only mashed. Meal samples had a final total volume of 400-800 mL before portioning aliquots of the meal to individual tubes to begin the digestive process. Digestion was initiated by the addition of pepsin and/or gliadinase Kuma062-M. Samples were then incubated at body temperature (37° C.) for the indicated timepoints. In most of the whole wheat bread/meal digestion experiments, samples were allowed to digest for 30 minutes, since the average lag time that food churns in the stomach before it begins to be released into the duodenum through the pyloric valve is 30-60 minutes. Enzyme activity was halted at the end of the digestion period by heating to a temperature that irreversibly inactivates all enzymes present.

Gluten in digestion samples was quantified by the R5 Ridascreen™ ELISA kit (R-Biopharm) or G12 Glutentox® ELISA kit (Biomedal), following the directions supplied by the manufacturer. These kits are based monoclonal antibodies, either R5 (recognizing QQPFP) or G12 (recognizing QPQLPY) (SEQ ID NO: 19 and SEQ ID NO: 20 respectively). These epitopes are present in most of the immunogenic fragments of gluten, including all of the immunodominant fragments. The G12 antibody detects the immunogenic region of α-gliadin, while the R5 antibody detects immunogenic regions of ω-gliadin and γ-gliadin. While the R5 ELISA method has been shown to be effective in estimating the gluten concentration of unprocessed foods, we have found that the fraction of gluten that is recognized by the R5 antibody is partially decreased following incubation of gluten with pepsin. Pepsin has been shown to be less effective against the fraction recognized by the G12 antibody, the 33mer fragment LQLQPFPQPQLPYPQPQLPYPQPQLPYPQPQPF8 (SEQ ID NO: 13). Unlike the R5 antibody, detection of gluten epitopes by the G12 antibody is frequently observed to be unaffected or even slightly increased by digestion with pepsin, suggesting that treatment with pepsin may make the QPQLPY (SEQ ID NO: 20) epitope-containing region of gluten more available to the G12 antibody. In this Example, both ELISA-based methods were used to assess the ability of gliadinase to decrease the amount of all three families of immunogenic gliadin: α-, ω-, and γ-gliadin. In one of the experiments detailed below, an in-house G12-based ELISA method was used. This in-house-developed method, while less expensive than the commercially available kits, is less reliable in quantification of low concentrations of gluten. Thus, this method was only used to assess relative differences between samples.

Table 6 shows that Kuma062-M can effectively degrade gluten in a simulated gastric digestion. Pepsin can degrade gluten in the simulated gastric digestion at a low level.

TABLE 6
Degradation of Gluten by Kuma062-M in Stimulated
Gastric Digestion*

| | | Gluten ppm | St | % | % |
|---|---|---|---|---|---|
| Enzyme | Timepoint | Remaining | Dev | Degraded | St Dev |
| Pepsin | 30 | 17920 | 640 | 4.55 | 3.41 |
| Kuma062-M | 5 | 200 | 14 | 98.93 | 0.07 |
| Kuma062-M | 30 | 48 | 4 | 99.75 | 0.02 |

Enzyme concentration: 100 μg/ml; Bread mixture: 16 mg/ml; St Dev: standard deviation

### Example 2: Degradation of Gluten in Whole Bread by Kuma062-M at Different pHs

This study is to evaluate the ability of Kuma062-M to degrade gluten at different pH values.

The protocol for the simulated gastric digestion is substantially similar to that in Example 1. Bread slurries were generated with the following pH levels: 3.9, 4.5, 5.0, 5.5, and 5.9. pH 5.9 was the pH of the bread slurry when only water, no HCl, was added to the slurry after mashing with artificial saliva.

Table 7 shows that Kuma062-M can degrade gluten effectively at various pH values.

TABLE 7
Degradation of Gluten by Kuma062-M at Different pH*

| | | | RS ELISA | | | | G12 ELISA | | | |
|---|---|---|---|---|---|---|---|---|---|---|
| | | | | | % of | | | | $ of | |
| Enzyme | | | Average | Standard | Gluten | Standard | Average | Standard | Gluten | Standard |
| Concentration | | pH | ppm | Dev | Degraded | Dev | ppm | Dev | Degraded | Dev |
| 1000 | ug/mL | 3.9 | 5.9 | 0.8 | 99.93% | 0.01% | 13.8 | 1.2 | 99.84% | 0.01% |
| | | 4.5 | 11.2 | 4.7 | 99.88% | 0.05% | 19.2 | 6.5 | 99.80% | 0.08% |
| | | 5.0 | 16.2 | 2.6 | 99.85% | 0.02% | 26.2 | 7.5 | 99.75% | 0.09% |
| | | 5.5 | 15.5 | 3.3 | 99.86% | 0.03% | 30.5 | 8.2 | 99.72% | 0.10% |
| | | 5.9 | 24.1 | 6.2 | 99.80% | 0.05% | 56.0 | 4.1 | 99.54% | 0.03% |
| 400 | ug/mL | 3.9 | 11.7 | 3.8 | 99.86% | 0.04% | 20.3 | 4.3 | 99.76% | 0.05% |
| | | 4.5 | 11.9 | 0.3 | 99.88% | 0.00% | 19.4 | 3.1 | 99.80% | 0.04% |
| | | 5.0 | 13.3 | 3.9 | 99.88% | 0.04% | 19.6 | 2.3 | 99.82% | 0.03% |
| | | 5.5 | 9.9 | 1.4 | 99.91% | 0.01% | 22.4 | 2.4 | 99.80% | 0.03% |
| | | 5.9 | 15.9 | 1.2 | 99.87% | 0.01% | 29.5 | 4.1 | 99.75% | 0.03% |
| 200 | ug/mL | 3.9 | 9.6 | 1.8 | 99.89% | 0.02% | 19.0 | 2.0 | 99.78% | 0.02% |

| | | | | | | | | |
|---|---|---|---|---|---|---|---|---|
| 4.5 | 12.4 | 4.5 | 99.87% | 0.05% | 22.8 | 3.2 | 99.76% | 0.04% |
| 5.0 | 5.5 | 0.8 | 99.95% | 0.01% | 25.0 | 6.7 | 99.77% | 0.08% |
| 5.5 | 11.7 | 1.9 | 99.89% | 0.02% | 24.9 | 2.6 | 99.77% | 0.03% |
| 5.9 | 15.3 | 1.5 | 99.87% | 0.01% | 37.5 | 5.0 | 99.69% | 0.04% |

Gluten concentration: 10 mg/ml

### Example 3: Degradation of Gluten in Fast Food Meal by Kuma062-M

This study is to evaluate whether Kuma062M is capable of maintaining significant activity against gluten even in the presence of other dietary protein.

The protocol for the simulated gastric digestion is substantially similar to that in Example 1. The vanilla milkshake was estimated (roughly, by comparisons to milkshakes of similar size from McDonalds®) to contain 10 grams of protein, while the hamburger patty was estimated to contain 7 grams of protein, pH of the meal in gastric digestion was 4.0-4.5. The amount of hamburger bun in the control meal was adjusted to the same amount of bun as in the hamburger and shake meal. Volume of gastric digestion of hamburger and shake meal was 500 mL; control meal was also adjusted to 500 mL. Aliquots of meal slurries after mashing and blending were portioned into smaller tubes, and glutenase enzyme and pepsin were added to these aliquots. Enzyme concentrations were 700 μg/mL or 70 μg/mL for Kuma062-M. Meal was digested for 30 minutes or 5 minutes. Aspergillus Niger-derived prolyl endoprotease (AN-PEP) and EPB2/SCPEP were also included in this study.

Tables 8 and 9 demonstrate that Kuma062-M can degrade gluten effectively in the presence of other dietary protein. Table 8 shows the result using G12 ELISA assay. Table 9 shows the results using R5 ELISA assay.

TABLE 8
Degradation of Gluten by Kuma062-M in Fast Food Meal G12 ELISA Assay

| | | | | Gluten | | | % | Equivalent | mg |
|---|---|---|---|---|---|---|---|---|---|
| | | | | (ppm) | St | % | St | mg | St |
| Enzyme | μg/ml | Timepoint | Meal | remaining | Dev | Degrade | Dev | remaining | Dev |
| Pepsin | 700 | 30 | Bun only | 13380 | 1004 | 8.03 | 6.41 | 6690 | 502 |
| Pepsin | 700 | 30 | Hamburger | 8056 | 464 | 55.61 | 2.54 | 4028 | 232 |
| ANPEP | 700 | 30 | Bun only | 434 | 23 | 97.06 | 0.15 | 217 | 12 |
| ANPEP | 700 | 30 | Hamburger | 4261 | 263 | 77.23 | 1.44 | 2131 | 132 |
| EP/SC | 700 | 30 | Bun only | 2394 | 97 | 85.08 | 0.62 | 1197 | 48 |
| EP/SC | 700 | 30 | Hamburger | 9401 | 940 | 54.47 | 5.15 | 4701 | 470 |
| Kuma062 | 700 | 5 | Bun only | 69 | 4 | 99.53 | 0.03 | 35 | 2 |
| Kuma062 | 700 | 30 | Bun only | 30 | 2 | 99.82 | 0.02 | 15 | 1 |
| Kuma062 | 700 | 5 | Hamburger | 83 | 7 | 99.59 | 0.04 | 42 | 4 |
| Kuma062 | 700 | 30 | Hamburger | 54 | 4 | 99.68 | 0.02 | 27 | 2 |
| Kuma062 | 70 | 30 | Bun only | 56 | 3 | 99.62 | 0.02 | 28 | 2 |
| Kuma062 | 70 | 30 | Hamburger | 151 | 6 | 99.14 | 0.03 | 75 | 3 |

TABLE 9
Degradation of Gluten by Kuma062-M in Fast Food Meal R5 ELISA Assay

| | | | | Gluten | | | % | Equivalent | mg |
|---|---|---|---|---|---|---|---|---|---|
| | | | | (ppm) | St | % | St | mg | St |
| Enzyme | μg/ml | Timepoint | Meal | remaining | Dev | Degrade | Dev | remaining | Dev |
| Pepsin | 700 | 30 | Bun only | 9680 | 554 | 38.27 | 3.53 | 4840 | 277 |
| Pepsin | 700 | 30 | Hamburger | 9493 | 1398 | 48.03 | 7.65 | 4747 | 699 |
| EP/SC | 700 | 30 | Bun only | 747 | 92 | 95.24 | 0.59 | 373 | 46 |
| EP/SC | 700 | 30 | Hamburger | 10400 | 604 | 43.07 | 3.31 | 5200 | 302 |
| Kuma062 | 700 | 5 | Bun only | 23 | 3 | 99.86 | 0.02 | 11 | 2 |
| Kuma062 | 700 | 30 | Bun only | 9 | 2 | 99.94 | 0.01 | 4 | 1 |
| Kuma062 | 700 | 5 | Hamburger | 113 | 17 | 99.38 | 0.09 | 56 | 9 |
| Kuma062 | 700 | 30 | Hamburger | 35 | 13 | 99.81 | 0.07 | 18 | 6 |
| Kuma062 | 70 | 30 | Bun only | 23 | 1 | 99.86 | 0.01 | 11 | 1 |
| Kuma062 | 70 | 30 | Hamburger | 147 | 6 | 99.20 | 0.03 | 73 | 3 |

### Equivalents

Those skilled in the art will recognize, or be able to ascertain using no more than routine experimentation, many equivalents to the specific aspects of the present disclosure. Such equivalents are intended to be encompassed by the following claims.

### ASPECTS

E1. A polypeptide comprising an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1.
E2. The polypeptide of E1, comprising an amino acid sequence having at least 85% sequence identity to the amino acid sequence set forth in SEQ ID NO: 1.
E3. The polypeptide of E1 or E2, comprising an amino acid sequence having at least 90% sequence identity to the amino acid sequence set forth in SEQ ID NO: 1.
E4. The polypeptide of any one of E1 to E3, comprising an amino acid sequence having at least 95% sequence identity to the amino acid sequence set forth in SEQ ID NO: 1.
E5. The polypeptide of any one of E1 to E4, comprising an amino acid sequence having at least 99% sequence identity to the amino acid sequence set forth in SEQ ID NO: 1.
E6. The polypeptide of any one of E1 to 5, comprising the amino acid sequence set forth in SEQ ID NO: 1.
E7. The polypeptide of any one of E1 to E6, wherein the amino acid residue corresponding to amino acid 467 of SEQ ID NO: 6 is a Ser.
E8. The polypeptide of any one of E1 to E7, wherein the amino acid residue corresponding to amino acid 267 of SEQ ID NO: 6 is a Glu.
E9. The polypeptide of any one of E1 to E8, wherein the amino acid residue corresponding to amino acid 271 of SEQ ID NO: 6 is an Asp.
E10. The polypeptide of any one of E1 to E9, which is capable of cleaving gliadin.
E11. A polypeptide comprising an amino acid sequence an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 8.
E12. The polypeptide of E11, comprising an amino acid sequence having at least 85% sequence identity to the amino acid sequence set forth in SEQ ID NO: 8.
E13. The polypeptide of E11 or E12, comprising an amino acid sequence having at least 90% sequence identity to the amino acid sequence set forth in SEQ ID NO: 8.
E14. The polypeptide of any one of E11 to E13, comprising an amino acid sequence having at least 95% sequence identity to the amino acid sequence set forth in SEQ ID NO: 8.
E15. The polypeptide of any one of E11 to 14, comprising an amino acid sequence having at least 99% sequence identity to the amino acid sequence set forth in SEQ ID NO: 8.
E16. The polypeptide of any one of E11 to E15, comprising the amino acid sequence set forth in SEQ ID NO: 8.
E17. The polypeptide of any one of E11 to E16, wherein the amino acid residue corresponding to amino acid 278 of SEQ ID NO: 3 is a Ser.
E18. The polypeptide of any one of E11 to E17, wherein the amino acid residue corresponding to amino acid 78 of SEQ ID NO: 3 is a Glu.
E19. The polypeptide of any one of E11 to E18, wherein the amino acid residue corresponding to amino acid 82 of SEQ ID NO: 3 is an Asp.
E20. The polypeptide of any one of E11 to E19, which is capable of cleaving gliadin.
E21. A polypeptide comprising an amino acid sequence an amino acid sequence having at least about 75%, at least about 80%, at least about 85%, at least about 90%, at least about 95%, at least about 96%, at least about 97%, at least about 98%, at least about 99%, or about 100% sequence identity to the amino acid sequence set forth in SEQ ID NO: 1: wherein the polypeptide comprises the amino acid sequence set forth in SEQ ID NO: 8.
E22. The polypeptide of E21, comprising an amino acid sequence having at least 85% sequence identity to the amino acid sequence set forth in SEQ ID NO: 1.
E23. The polypeptide of E21 or E22, comprising an amino acid sequence having at least 90% sequence identity to the amino acid sequence set forth in SEQ ID NO: 1.
E24. The polypeptide of any one of E21 to E23, comprising an amino acid sequence having at least 95% sequence identity to the amino acid sequence set forth in SEQ ID NO: 1.
E25. The polypeptide of any one of E21 to 24, comprising an amino acid sequence having at least 99% sequence identity to the amino acid sequence set forth in SEQ ID NO: 1.
E26. The polypeptide of any one of E21 to E25, comprising the amino acid sequence set forth in SEQ ID NO: 1.
27. The polypeptide of any one of E21 to E26, wherein the amino acid residue corresponding to amino acid 467 of SEQ ID NO: 6 is a Ser.
E28. The polypeptide of any one of E21 to E27, wherein the amino acid residue corresponding to amino acid 267 of SEQ ID NO: 6 is a Glu.
E29. The polypeptide of any one of E21 to E28, wherein the amino acid residue corresponding to amino acid 271 of SEQ ID NO: 6 is an Asp.
E30. The polypeptide of any one of E21 to E29, which is capable of cleaving gliadin.
E31. The polypeptide of any one of E1 to E30, further comprising a histidine tag, wherein the histidine tag is fused at the C-terminus of the polypeptide.
E32. The polypeptide of E, wherein the histidine tag comprises the amino acid sequence set forth in SEQ ID NO: 17 (GSTENLYFQSGALEHHHHHH).
E33. The polypeptide of E32 or E33, wherein the histidine tag comprises a cleavable histidine tag, including but not limited to a cleavable histidine tag comprising the amino acid sequence set forth in SEQ ID NO: 15 (X_{N}PQ(L/Q)PX_{N}HHHHHH), wherein X_{N} is an linker of between 1-25 amino acid residues.
E34. The polypeptide of any one of E31 to E33, wherein the cleavable histidine tag comprises the amino acid sequence set forth in SEQ ID NO: 16 (GSSGSSGSQPQLPYGSSGSSGSHHHHHH).
E35. A nucleic acid molecule encoding the polypeptide of any one of E1 to E34.
E36. A nucleic acid expression vector comprising the nucleic acid molecule of E35.
E37. A recombinant host cell comprising the nucleic acid molecule of E35 or the nucleic acid expression vector of E36.
E38. A pharmaceutical composition, comprising the polypeptide of any one of E1 to E34, the nucleic acid molecule of E35, the nucleic acid expression vector of E36, the recombinant host cell of E37, or any combination thereof and a pharmaceutically acceptable carrier.
E39. A method for treating celiac sprue or non-celiac gluten sensitivity (NCGS), comprising administering to an individual with celiac sprue or NCGS an amount effective to treat the celiac sprue or NCGS of the polypeptide of any one of E1 to E34, the nucleic acid molecule of claim 35, the nucleic acid expression vector of claim 36, the recombinant host cell of claim 37, or the pharmaceutical composition of claim 38.
E40. The method of E39, wherein the polypeptide, the nucleic acid molecule, the nucleic acid expression vector, the recombinant host cell, or the pharmaceutical composition is administered orally.

1. A polypeptide comprising an amino acid sequence having at least about 75% sequence identity to the amino acid sequence set forth in SEQ ID NO: 1, wherein the first amino acid at the N-terminus of the polypeptide is a Ser (S).

2. (canceled)

3. A polypeptide comprising an amino acid sequence having at least about 75% sequence identity to the amino acid sequence set forth in SEQ ID NO: 23, wherein the Xaa in SEQ ID NO: 23 is not a Met (M).

4. A polypeptide comprising an amino acid sequence having at least about 75% sequence identity to the amino acid sequence set forth in SEQ ID NO: 1, wherein the first amino acid at the N-terminus of the polypeptide is a Ser (S); wherein the polypeptide comprises the amino acid sequence set forth in SEQ ID NO: 8.

5. The polypeptide of claim 1, wherein the first two N-terminal amino acids of the polypeptide, from N-terminus to C-terminus, are Ser-Asp (SD).

6.-7. (canceled)

8. The polypeptide of claim 1, comprising an amino acid sequence having at least 95% sequence identity to the amino acid sequence set forth in SEQ ID NO: 1.

9. The polypeptide of claim 1, comprising an amino acid sequence having at least 99% sequence identity to the amino acid sequence set forth in SEQ ID NO: 1.

10. The polypeptide of claim 1, comprising the amino acid sequence set forth in SEQ ID NO: 1.

11. The polypeptide of claim 1, wherein the amino acid residue corresponding to amino acid 467 of SEQ ID NO: 1 is a Ser.

12. The polypeptide of claim 1, wherein the amino acid residue corresponding to amino acid 267 of SEQ ID NO: 1 is a Glu.

13. The polypeptide of claim 1, wherein the amino acid residue corresponding to amino acid 271 of SEQ ID NO: 1 is an Asp.

14. (canceled)

15. The polypeptide of claim 1, further comprising a histidine tag, wherein the histidine tag is fused at the C-terminus of the polypeptide, wherein the histidine tag comprises the amino acid sequence set forth in SEQ ID NO: 17 (GSTENLYFQSGALEHHHHHH).

16. (canceled)

17. The polypeptide of claim 15 , wherein the histidine tag comprises a cleavable histidine tag comprising the amino acid sequence set forth in SEQ ID NO: 15 (X_{N}PQ(L/Q)PX_{N}HHHHHH), wherein X_{N} is an linker of between 1-25 amino acid residues.

18. The polypeptide of claim 17, wherein the cleavable histidine tag comprises the amino acid sequence set forth in SEQ ID NO: 16 (GSSGSSGSQPQLPYGSSGSSGSHHHHHH).

19. A nucleic acid molecule encoding the polypeptide of claim 1.

20. A nucleic acid expression vector comprising the nucleic acid molecule of claim 19.

21. A recombinant host cell comprising the nucleic acid molecule of claim 19.

22.-23. (canceled)

24. A pharmaceutical composition comprising the polypeptide of claim 1 and a pharmaceutically acceptable carrier.

25. A method for treating celiac sprue or non-celiac gluten sensitivity (NCGS) in a subject, comprising administering to the subject with celiac sprue or NCGS an amount effective to treat the celiac sprue or NCGS of the pharmaceutical composition of claim 24, thereby treating the celiac sprue or NCGS.

26. A method for reducing celiac sprue or non-celiac gluten sensitivity (NCGS) related inflammation in a subject, comprising orally administering to the subject with celiac sprue or NCGS an amount effective to reduce the celiac sprue or NCGS related inflammation the pharmaceutical composition of claim 24, thereby reducing the inflammation.

27. (canceled)

28. A method for degrading gluten in a food item, comprising contacting the food item with an amount effective to degrade the gluten with the polypeptide of claim 1.

29.-30. (canceled)
